SEQUENCE LISTING (44 sequences)

SEQ ID NO: 1 - protein, 13 aa, Artificial Sequence
  Synthetic; compstatin
ICVVQDWGHHRCT

SEQ ID NO: 2 - protein, 42 aa, Artificial Sequence
  Synthetic
XXXXXXXXXXXXXXXXXXXQDXGXXXXXXXXXXXXXXXXXXX

SEQ ID NO: 3 - protein, 5 aa, Artificial Sequence
  Synthetic; core sequence of peptide
XQDXG

SEQ ID NO: 4 - protein, 6 aa, Artificial Sequence
  Synthetic
XQDXGX

SEQ ID NO: 5 - protein, 13 aa, Artificial Sequence
  Synthetic
XXXXQDXGXXXXX

SEQ ID NO: 6 - protein, 16 aa, Artificial Sequence
  Synthetic
XXCVXQDXGXHRCXXX

SEQ ID NO: 7 - protein, 16 aa, Artificial Sequence
  Synthetic
XXCVXQDXGXHRCXXX

SEQ ID NO: 8 - protein, 13 aa, Artificial Sequence
  Synthetic; compstatin
ICVVQDWGHHRCT

SEQ ID NO: 9 - protein, 13 aa, Artificial Sequence
  Synthetic; compstatin analog
ICVVQDWGHHRCT

SEQ ID NO: 10 - protein, 13 aa, Artificial Sequence
  Synthetic; compstatin analog
ICVYQDWGAHRCT

SEQ ID NO: 11 - protein, 13 aa, Artificial Sequence
  Synthetic; compstatin analog
ICVWQDWGAHRCT

SEQ ID NO: 12 - protein, 13 aa, Artificial Sequence
  Synthetic; compstatin analog
ICVWQDWGAHRCT

SEQ ID NO: 13 - protein, 13 aa, Artificial Sequence
  Synthetic; compstatin analog
ICVWQDWGAHRCT

SEQ ID NO: 14 - protein, 13 aa, Artificial Sequence
  Synthetic; compstatin analog
ICVAQDWGAHRCT

SEQ ID NO: 15 - protein, 13 aa, Artificial Sequence
  Synthetic; compstatin analog
ICVAQDWGAHRCT

SEQ ID NO: 16 - protein, 13 aa, Artificial Sequence
  Synthetic; compstatin analog
ICVAQDWGAHRCT

SEQ ID NO: 17 - protein, 13 aa, Artificial Sequence
  Synthetic; compstatin analog
ICVGQDWGAHRCT

SEQ ID NO: 18 - protein, 13 aa, Artificial Sequence
  Synthetic; compstatin analog
ICVGQDWGAHRCT

SEQ ID NO: 19 - protein, 13 aa, Artificial Sequence
  Synthetic; compstatin analog
ICVWQDWGAHRCT

SEQ ID NO: 20 - protein, 13 aa, Artificial Sequence
  Synthetic; compstatin analog
ICVFQDWGAHRCT

SEQ ID NO: 21 - protein, 13 aa, Artificial Sequence
  Synthetic; compstatin analog
ICVFQDWGAHRCT

SEQ ID NO: 22 - protein, 13 aa, Artificial Sequence
  Synthetic; compstatin analog
ICVAQDWGAHRCT

SEQ ID NO: 23 - protein, 13 aa, Artificial Sequence
  Synthetic; compstatin analog
ICVAQDWGAHRCT

SEQ ID NO: 24 - protein, 13 aa, Artificial Sequence
  Synthetic; compstatin analog
ICVWQDWGXHRCT

SEQ ID NO: 25 - protein, 16 aa, Artificial Sequence
  Synthetic; compstatin analog
GICVWQDWGAHRCTAN

SEQ ID NO: 26 - protein, 13 aa, Artificial Sequence
  Synthetic; compstatin analog
ICVWQDWGAHRCT

SEQ ID NO: 27 - protein, 13 aa, Artificial Sequence
  Synthetic; compstatin analog
ICVWQDWGAHRCT

SEQ ID NO: 28 - protein, 13 aa, Artificial Sequence
  Synthetic; compstatin analog
ICVWQDWGAHRCT

SEQ ID NO: 29 - protein, 13 aa, Artificial Sequence
  Synthetic; compstatin analog
ICVWQDWGAHRCT

SEQ ID NO: 30 - protein, 13 aa, Artificial Sequence
  Synthetic; compstatin analog
ICVWQDWGAHRCT

SEQ ID NO: 31 - protein, 13 aa, Artificial Sequence
  Synthetic; compstatin analog
ICVWQDWGAHRCT

SEQ ID NO: 32 - protein, 13 aa, Artificial Sequence
  Synthetic; compstatin analog
ICVWQDWGAHRCT

SEQ ID NO: 33 - protein, 15 aa, Artificial Sequence
  Synthetic; compstatin analog
GICVWQDWGAHRCTN

SEQ ID NO: 34 - protein, 13 aa, Artificial Sequence
  Synthetic; compstatin analog
ICVWQDWGAHRCT

SEQ ID NO: 35 - protein, 13 aa, Artificial Sequence
  Synthetic; compstatin analog
ICVWQDWGAHRCT

SEQ ID NO: 36 - protein, 15 aa, Artificial Sequence
  Synthetic; compstatin analog
GICVWQDWGAHRCTN

SEQ ID NO: 37 - protein, 5 aa, Artificial sequence
  Synthetic peptide
XPXWR

SEQ ID NO: 38 - protein, 5 aa, Artificial sequence
  Synthetic peptide
KPXWR

SEQ ID NO: 39 - protein, 10 aa, Artificial sequence
  Synthetic peptide
XXXXXXPXWR

SEQ ID NO: 40 - protein, 10 aa, Artificial sequence
  Synthetic peptide
XXXXXKPXWR

SEQ ID NO: 41 - protein, 6 aa, Artificial sequence
  Synthetic peptide
FXPXWR

SEQ ID NO: 42 - protein, 6 aa, Artificial sequence
  Synthetic peptide
FKPXWR

SEQ ID NO: 43 - protein, 5 aa, Artificial sequence
  Synthetic peptide
XPXWR

SEQ ID NO: 44 - protein, 5 aa, Artificial sequence
  Synthetic peptide
XPXWR

(19) United States
(12) Patent Application Publication
(10) Pub. No.: US 2011/0182877 A1
(43) Pub. Date: Jul. 28, 2011

(54) SUSTAINED DELIVERY OF COMPSTATIN ANALOGS FROM GELS

(75) Inventors: Cedric Francois, Louisville, KY (US); Pascal Deschatelets, Louisville, KY (US); Paul Olson, Louisville, KY (US)

(73) Assignee: POTENTIA PHARMACEUTICALS, INC., Louisville, KY (US)

(21) Appl. No.: 12/681,392

(22) PCT Filed: Oct. 2, 2008
(86) PCT No.: PCT/US08/78593

§ 371 Date: Apr. 2, 2010

Related U.S. Application Data
(60) Provisional application No. 60/976,919, filed on Oct. 2, 2007.
(60) Provisional application No. 61/026,460, filed on Feb. 5, 2008.

(51) Int. Cl. A61K 38/08; A61K 39/395; A61K 38/10; A61P 27/02

(52) U.S. Cl. 424/130.1; 514/21.8; 514/21.5; 514/21.4

(57) ABSTRACT

The present invention features the sustained delivery of compstatin analog and, optionally, an additional active agent, by release from a macroscopic, gel-like deposit formed by administering a liquid solution containing the compstatin analog to an extravascular location in the body of a mammalian subject such as the vitreous chamber.

### CROSS-REFERENCE TO RELATED APPLICATIONS

This application claims priority to, and the benefit of, U.S. Provisional Patent Application No. U.S. Ser. No. 60/976,919, filed Oct. 2, 2007, and U.S. Ser. No. 61/026,460, filed Feb. 5, 2008. The contents of these applications are incorporated herein by reference.

### BACKGROUND OF THE INVENTION

The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated Cl cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils.

The alternative pathway is initiated by microbial surfaces and various complex polysaccharides. In this pathway, C3b, resulting from cleavage of C3, which occurs spontaneously at a low level, binds to targets, e.g., on cell surfaces and forms a complex with factor B, which is later cleaved by factor D, resulting in a C3 convertase. Cleavage of C3 and binding of another molecule of C3b to the C3 convertase gives rise to a C5 convertase. C3 and C5 convertases of this pathway are regulated by CR1, DAF, MCP, and fH. The mode of action of these proteins involves either decay accelerating activity (i.e., ability to dissociate convertases), ability to serve as cofactors in the degradation of C3b or C4b by factor I, or both.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains. These domains, consisting of approximately 50-70 amino acids, typically about 60 amino acids, are characterized by a conserved motif that includes four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4bp). CD59 is a membrane-bound complement regulator unrelated structurally to the CCPs.

Further details regarding the complement system and its activation pathways are found in the following references: Makrides, S C, Pharm Rev., 50(1): 59-87, 1998; Lisczewski, M K and Atkinson, J P, in The Human Complement System in Health and Disease, Volanakis, J E and Frank, M M, eds., Dekker, New York, pp. 149-66, 1998; Kuby Immunology, 2000; Paul, W. E., Fundamental Immunology, Lippincott Williams & Wilkins; 5^{th} ed., 2003; and Walport M. J., Complement. First of two parts. N Engl J Med., 344(14):1058-66, 2001.

While complement activation plays important roles in the innate and adaptive immune systems, the complement system is increasingly recognized to be involved in tissue injury during a variety of ischemic, inflammatory, and autoimmune diseases (Makrides, S C, Pharm Rev., 50(1): 59-87, 1998; Lisczewski, M K and Atkinson, J P, in The Human Complement System in Health and Disease, Volanakis, J E and Frank, M M, eds., Dekker, New York, pp. 149-66, 1998). Complement inhibition has been proposed as a therapeutic strategy for many such diseases. Compstatin and its analogs are cyclic peptides that bind to C3 and inhibit its activation. There is a need in the art for new compositions and methods for administering compstatin analogs. There is also a need in the art for improved drug delivery systems.

### SUMMARY OF THE INVENTION

The present invention provides novel formulations and methods for sustained release administration of a compstatin analog to a mammalian subject. In one aspect, the invention provides a liquid composition comprising a compstatin analog in an amount sufficient to form a macroscopic gel-like structure when introduced into an extravascular location in the body of a mammalian subject. In certain embodiments of the invention the extravascular location is the vitreous chamber. In other embodiments, the extravascular location is the subconjunctival space. In some embodiments, the extravascular location is the retrobulbar, subconjunctival, sub-Tenon's, or subretinal space. In certain embodiments of the invention the compstatin analog has an activity at least 100-fold greater than that of compstatin. In certain embodiments of the invention the compstatin analog has an activity at least 150-fold greater than that of compstatin. In certain embodiments of the invention the compstatin analog has an activity at least 200-fold greater than that of compstatin. In certain embodiments of the invention the compstatin analog has an activity at least 250-fold greater than that of compstatin. In certain embodiments of any of the methods of the invention involving administration to a subject, the invention the subject is a non-human primate. In certain embodiments of any of the methods of the invention involving administration to a subject, the subject is a human.

In some embodiments the liquid composition comprises a compstatin analog and a second active agent in addition to the compstatin analog. The second active agent may be a polypeptide, peptide, non-peptidic small molecule, nucleic acid, etc. In some embodiments the second active agent is a complement inhibitor. In some embodiments the second active agent is an angiogenesis inhibitor.

The invention further provides a method of treating a complement-mediated disorder in a mammalian subject comprising administering any of the afore-mentioned liquid compositions to the subject. In one aspect, the invention provides a method of treating a complement-mediated disorder comprising the step of: administering a liquid composition comprising an effective amount of a compstatin analog to an extravascular location in the body of a subject, wherein said effective amount is sufficient to form a macroscopic gel-like structure containing the compstatin analog within said extravascular location. In certain embodiments said effective amount is sufficient to form a macroscopic gel-like structure that diminishes in size over time and remains readily detectable for at least 2 weeks. In certain embodiments the macroscopic gel-like structure diminishes in size over time and releases the compstatin analog in active form so as to achieve a therapeutic concentration in the extravascular location for at least 2 weeks, at least 4 weeks, at least 2 months, at least 3 months, at least 6 months, at least 9 months, or at least 12 months. In certain embodiments the macroscopic gel-like structure remains readily detectable for at least 2 weeks, at least 4 weeks, at least 2 months, at least 3 months, at least 6 months, at least 9 months, or at least 12 months, e.g., up to about 18 or about 24 months. A compstatin analog in “active form” retains the ability to bind to C3 and inhibit its cleavage.

In some embodiments the composition is administered to the vitreous chamber of a subject suffering from or at risk of age-related macular degeneration (AMD). In some embodiments the subject suffers from or is at risk of dry AMD. In some embodiments, the subject suffers from or is at risk of diabetic retinopathy, uveitis, glaucoma, or retinitis pigmentosa.

In some embodiments the composition is administered to the intrathecal cavity of a subject. The subject may suffer from a spinal cord injury or chronic pain.

In some embodiments the composition is administered to the cranial cavity of a subject, e.g., into a ventricle. The subject may suffer from multiple sclerosis, Parkinson's disease, Alzheimer's disease, or stroke.

In some embodiments the composition is administered to a synovial cavity or bursa of a subject. The subject may suffer from arthritis, e.g., rheumatoid arthritis, psoriatic arthritis, Reiter's syndrome, juvenile arthritis, or gout.

In another aspect, methods for making the compositions of the invention are also provided.

In some aspects, the invention provides a method of treating a complement-mediated disorder comprising the step of administering a liquid composition comprising an effective amount of a compstatin analog to an extravascular location of a subject, wherein said effective amount is sufficient to form a discrete, macroscopic gel-like structure containing the compstatin analog within said extravascular location. In some embodiments, the effective amount is sufficient to form a macroscopic gel-like structure that diminishes in size over time and remains readily detectable for at least 2 weeks. In some embodiments, the effective amount is sufficient to form a macroscopic gel-like structure that diminishes in size over time and releases compstatin analog in active form for at least 2 weeks. In some embodiments, the effective amount is sufficient to form a macroscopic gel-like structure that diminishes in size over time and remains readily detectable for at least 3 months. In some embodiments, the effective amount is sufficient to form a macroscopic gel-like structure that diminishes in size over time and releases compstatin analog in active form for at least 3 months. In some embodiments, the effective amount is sufficient to form a macroscopic gel-like structure that diminishes in size over time and releases the compstatin analog in active form so as to achieve a therapeutic concentration of said compstatin analog within the extravascular location or nearby tissue for at least 2 weeks. In some embodiments, the effective amount is sufficient to form a macroscopic gel-like structure that diminishes in size over time and releases the compstatin analog in active form so as to achieve a therapeutic concentration of said compstatin analog within the extravascular location or nearby tissue for at least 3 months. In some embodiments, the compstatin analog comprises a peptide whose sequence comprises a sequence selected from the group consisting of SEQ ID NOs: 3, 4, 5, 6, and 7. In some embodiments, the compstatin analog has at least 100-fold greater activity than SEQ ID NO: 8. In some embodiments, the compstatin analog has at least 200-fold greater activity than SEQ ID NO: 8. In some embodiments, the compstatin analog has a sequence selected from SEQ ID NOs: 14, 21, 28, 29, 30, 31, 32, 33, 34, and 36. In some embodiments, the compstatin analog has a sequence selected from SEQ ID NOs: 28, 32, and 34. In some embodiments the compstatin analog is the peptide of SEQ ID NO: 14. In some embodiments the compstatin analog is the peptide of SEQ ID NO: 28. In some embodiments the compstatin analog is the peptide of SEQ ID NO: 30. In some embodiments the compstatin analog is the peptide of SEQ ID NO: 32. In some embodiments the compstatin analog is the peptide of SEQ ID NO: 33. In some embodiments the compstatin analog is the peptide of SEQ ID NO: 34. In some embodiments, the amount of said compstatin analog in the liquid composition is between 1 mg/ml and 50 mg/ml. In some embodiments, the amount of said compstatin analog in the liquid composition is between 2 mg/ml and 25 mg/ml. In some embodiments, between 50 μg and 5000 μg of compstatin analog is administered to the vitreous chamber. In some embodiments, between 150 μg and 2000 μg of compstatin analog is administered to the vitreous chamber. In some embodiments, between 400 μg and 1500 μg of compstatin analog is administered to the vitreous chamber. In some embodiments, about 450 μg compstatin analog is administered to the vitreous chamber. In some embodiments, about 1050 μg compstatin analog is administered to the vitreous chamber. In some embodiments, the compstatin analog is administered to the vitreous chamber in a volume between 25 μl and 125 μl. In some embodiments, the compstatin analog is administered to the vitreous chamber in a volume of about 50 μl. In some embodiments, the compstatin analog is administered to the vitreous chamber in a volume of about 75 μl. In some embodiments, the subject suffers from age related macular degeneration and the liquid composition is administered to the vitreous chamber. In some embodiments, the liquid composition further comprises an effective amount of a second therapeutic agent. In some embodiments, the the second therapeutic agent is a complement inhibitor, angiogenesis inhibitor, steroid, anti-inflammatory agent, anti-infective, or analgesic. In some embodiments, the composition is administered by intravitreal injection. In some embodiments, the composition comprises a plurality of microparticles or nanoparticles. The microparticles or nanoparticles may comprise a therapeutic agent, which may, but need not be, be a compstatin analog, and if it is a compstatin analog may, but need not be, the same compstatin analog as that which forms the gel. In some embodiments, at least some of the microparticles or nanoparticles become trapped in the gel upon administration.

The invention provides a gel-like structure comprising a compstatin analog and at least one endogenous polypeptide normally present in an extravascular location of a subject. In some embodiments the polypeptide is one that is present in an extravascular location selected from the group consisting of: the vitreous chamber, subconjunctival space, sub-Tenon's space, subretinal space, synovial cavity, and the cerebrospinal cavity.

The invention provides a liquid composition comprising a compstatin analog, wherein the composition is characterized in that it forms a macroscopic, gel-like structure when administered to the vitreous chamber of a mammalian subject. In some embodiments the compstatin analog in present in an amount sufficient to form a discrete, macroscopic gel-like structure when administered to the vitreous chamber of the subject by intravitreal injection, e.g., in a volume of about 50 μl to about 100 μl. In some embodiments the compstatin analog is present in an amount sufficient to form a macroscopic gel-like structure that diminishes in size over time and remains readily detectable for at least 2 weeks. In some embodiments the compstatin analog is present in an amount sufficient to form a macroscopic gel-like structure that diminishes in size over time and releases compstatin analog in active form for at least 2 weeks. In some embodiments the compstatin analog is present in an amount sufficient to form a macroscopic gel-like structure that diminishes in size over time and remains readily detectable for at least 3 months. In some embodiments the compstatin analog is present in an amount sufficient to form a macroscopic gel-like structure that diminishes in size over time and releases compstatin analog in active form for at least 3 months. In some embodiments the compstatin analog is present in an amount sufficient to form a macroscopic gel-like structure that diminishes in size over time and remains readily detectable for at least 6 months. In some embodiments the compstatin analog is present in an amount sufficient to form a macroscopic gel-like structure that diminishes in size over time and releases active compstatin analog for at least 6 months. In some embodiments the compstatin analog is present in an amount sufficient to form a macroscopic gel-like structure that diminishes in size over time and releases the compstatin analog in active form so as to achieve a therapeutic concentration of said compstatin analog within the the vitreous chamber or nearby tissue for at least 2 weeks. In some embodiments the compstatin analog is present in an amount sufficient to form a macroscopic gel-like structure that diminishes in size over time and releases the compstatin analog in active form so as to achieve a therapeutic concentration of said compstatin analog within the vitreous chamber or nearby tissue for at least 3 months. In some embodiments the compstatin analog comprises a peptide whose sequence comprises a sequence selected from the group consisting of SEQ ID NOs: 3, 4, 5, 6, and 7. In some embodiments the compstatin analog has at least 100-fold greater activity than SEQ ID NO: 8. In some embodiments the compstatin analog has at least 200-fold greater activity than SEQ ID NO: 8. In some embodiments the sequence of the compstatin analog comprises a sequence selected from SEQ ID NOs: 14, 21, 28, 29, 30, 31, 32, 33, 34, and 36. In some embodiments the sequence of the compstatin analog comprises a sequence selected from SEQ ID NOs: 28, 32, and 34. In some embodiments the sequence of the compstatin analog comprises SEQ ID NO: 28. In some embodiments the sequence of the compstatin analog comprises SEQ ID NO: 32. In some embodiments the sequence of the compstatin analog comprises SEQ ID NO: 34. In some embodiments the amount of said compstatin analog in the liquid composition is between 1 mg/ml and 50 mg/ml. In some embodiments the amount of said compstatin analog in the liquid composition is between 3 mg/ml and 25 mg/ml. In some embodiments the composition contains between 150 μg and 5000 μg of compstatin analog. In some embodiments the composition contains between 250 μg and 2000 μg of compstatin analog. In some embodiments the composition contains between 400 μg and 1500 μg of compstatin analog. In some embodiments the composition has a volume between 25 μl and 125 μl. In some embodiments the composition contains between 150 μg and 2000 μg of compstatin analog in a volume between 50 μl and 100 μl. In some embodiments the composition consists essentially of compstatin analog in water. In some embodiments the composition is substantially free of excipients. In some embodiments the composition comprises a component selected from the group consisting of: sugar alcohols and amino acids. In some embodiments the presence of the component modulates the rate at which the deposit disappears in vivo. In some embodiments the composition comprises histidine. In some embodiments the composition comprises a buffer. In some embodiments n the composition comprises sodium acetate. In some embodiments the composition comprises mannitol. In some embodiments the liquid composition further comprises an effective amount of a second therapeutic agent. In some embodiments the second therapeutic agent is a complement inhibitor, angiogenesis inhibitor, steroid, anti-inflammatory agent, anti-infective, or analgesic.

The invention also provides a method of preparing a composition for delivery of a therapeutic agent over a sustained period of time comprising: preparing a liquid composition comprising the therapeutic agent and a compstatin analog, wherein the compstatin analog is present in sufficient amounts to form a macroscopic, gel-like structure when the composition is administered to an extravascular location of a mammalian subject. In some embodiments the method further comprises administering the liquid composition to an extravascular location in the body of a mammalian subject. In some embodiments the extravascular location is the vitreous chamber. In some embodiments the extravascular location is the vitreous chamber and the subject suffers from AMD.

In some aspects, the invention provides a method of treating a subject suffering from or at risk of AMD comprising administering a liquid composition comprising a compstatin analog directly to the subject's vitreous chamber, wherein the liquid composition contains a sufficient amount of the compstatin analog to form a macroscopic, gel-like structure following administration. In certain embodiments the compstatin analog comprises a peptide whose sequence comprises a sequence selected from the group consisting of SEQ ID NOs: 3, 4, 5, 6, and 7. In certain embodiments the compstatin analog has at least 100-fold greater activity than SEQ ID NO: 8. In certain embodiments the compstatin analog has at least 200-fold greater activity than SEQ ID NO: 8. In certain embodiments the compstatin analog has a sequence selected from SEQ ID NOs: 14, 21, 28, 29, 30, 31, 32, 33, 34, and 36. In certain embodiments the compstatin analog has a sequence selected from SEQ ID NOs: 28, 32, and 34. In certain embodiments the amount of the compstatin analog is between 2 mg/ml and 20 mg/ml. In certain embodiments between 100 μg and 2,000 μg compstatin analog is administered to the eye. In certain embodiments between 250 μg and 1,500 μg compstatin analog is administered to the eye. In certain embodiments between 400 μg and 1,200 μg compstatin analog is administered to the eye. In certain embodiments the liquid composition further comprises an angiogenesis inhibitor.

The invention provides liquid composition comprising a compstatin analog and water, wherein the concentration of the compstatin analog is between 3 and 50 mg/ml. In certain embodiments the concentration of the compstatin analog is between 5 and 30 mg/ml. In certain embodiments the concentration of the compstatin analog is between 8 and 25 mg/ml. In certain embodiments of any of such compositions, the compstatin analog comprises a peptide selected from the group consisting of SEQ ID NOs: 3, 4, 5, 6, and 7. In certain embodiments of any of such compositions, the compstatin analog has at least 100-fold greater activity than SEQ ID NO: 8. In certain embodiments of any of such compositions, the compstatin analog has at least 200-fold greater activity than SEQ ID NO: 8. In certain embodiments of any of such compositions, the compstatin analog has a sequence selected from SEQ ID NOs: 14, 21, 28, 29, 30, 31, 32, 33, 34, and 36. In certain embodiments of any of such compositions, compstatin analog has a sequence selected from SEQ ID NOs: 28, 32, and 34. In certain embodiments of any of such compositions, the composition consists essentially of the compstatin analog and water. In certain embodiments of any of such compositions, the composition further comprises an excipient selected from amino acids and sugar alcohols. The invention provides liquid composition comprising a compstatin analog and water, wherein the concentration of the compstatin analog is between 100 and 2000 mg/ml, e.g., between 100 and 1000 mg/ml, or between 100 and 500 mg/ml, wherein the composition comprises a component that modifies the properties of the composition so that a gel formed upon administration to an extravascular location (e.g., the vitreous chamber) degrades or disintegrates more rapidly than had the component not been present. In some embodiments, the component is an excipient selected from a sugar alcohol and an amino acid. In some embodiments the amino acid is a standard amino acid, e.g., histidine. In some embodiments the sugar alcohol is mannitol. In some embodiments the component is a buffer, e.g., sodium acetate.

Unless otherwise stated, the invention makes use of standard methods of molecular biology, chemistry, cell culture, animal maintenance, medical and veterinary examination, etc., and uses art-accepted meanings of terms. This application refers to various patents and publications. The contents of all scientific articles, books, patents, patent applications, and other publications, mentioned in this application are incorporated herein by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kuby Immunology, 4^{th} ed., Goldsby, R. A., Kindt, T. J., and Osborne, B. (eds.), W.H. Freeman, 2000, Goodman and Gilman's The Pharmacological Basis of Therapeutics, 10^{th} Ed., McGraw Hill, 2001, Katzung, B. (ed.) Basic and Clinical Pharmacology, McGraw-Hill/Appleton & Lange; 9th edition (December 2003); Goldman & Ausiello, Cecil Textbook of Medicine, 22^{nd} ed., W.B. Saunders, 2003. In the event of a conflict or inconsistency between any of the incorporated references and the instant specification, the specification (including any amendments thereto) shall control. Art-accepted abbreviations for the amino acids are used herein unless otherwise indicated.

### BRIEF DESCRIPTION OF THE DRAWING

FIG. 1 shows a macroscopic gel-like structure removed from the vitreous of a rabbit that had been administered a potent compstatin analog by intravitreal injection.

FIG. 2 shows B-scan ultrasound images of animals 12 days (left) and 8 weeks (right) post injection of a potent compstatin analog. The deposit is prominent 12 days following injection and much smaller but still detectable after 8 weeks.

FIG. 3 is an SDS-PAGE gel stained to show proteins present in the deposit together with a potent compstatin analog. Proteins found in vitreous with and without compstatin analog injected are also shown.

FIG. 4 is a plot showing that the compstatin analog present in the deposits remains stable and retains complement inhibiting activity over time. Blue squares: Compstatin analog (standard material); Green squares: Compstatin analog obtained from gel removed from rabbit vitreous 6 weeks after injection; Brown diamonds: Compstatin analog obtained from gel removed from rabbit vitreous 8 months after injection; Red ovals: Inactive control peptide G8A.

FIG. 5 is a plot showing measured concentration of a compstatin analog in serum and vitreous of Cynomolgus monkeys 14 days following intravitreal injection with 0, 450, 1050, or 2100 μg of the compound. All values are expressed as mean±standard error; n=4 for each vitreous and n=8 for serum. Note that the actual values for serum are 100-fold lower than shown, i.e., approximately 100-fold lower than vitreal concentration at the same administered dose.

### DEFINITIONS

Before describing the present invention in detail, it is to be understood that this invention is not limited to particularly exemplified systems or parameters as such may, of course, vary. It is also to be understood that the terminology used herein is for the purpose of describing particular embodiments of the invention only, and is not intended to limit the scope of the invention in any manner. The definitions below are provided for the convenience of the reader and are not intended to conflict with the usage of such terms in the art unless specifically indicated.

The terms “angiogenesis inhibitor” and “antiangiogenic agent” are used interchangeably herein to refer to agents that are capable of inhibiting or reducing one or more processes associated with formation, growth, and/or development of new blood vessels including, but not limited to, endothelial cell proliferation, endothelial cell migration, and capillary tube formation. In addition, such agents may inhibit fluid exudation from blood vessels.

The term “antagonist” refers to a compound which inhibits (e.g., antagonizes, reduces, decreases, blocks, or reverses) the effect of a given molecule. An antagonist is capable of acting in a manner relative to a particular molecule's activity, such that the biological activity of the molecule is decreased or blocked in a manner that is antagonistic (e.g., against, opposite to, contrary to) to one or more natural actions of the molecule. Antagonists can include, but are not limited to, an antibody or antigen binding fragment thereof, a protein, peptide, nucleic acid (such as RNAi agents, ribozymes, and antisense), or a small molecule.

The term “antibody” refers to an immunoglobulin or a derivative thereof containing an an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value).

“Biocompatible” is used consistently with its usage in the art and refers to a material that is substantially non-toxic to cells in vitro, e.g., in certain embodiments if its addition to cells in culture in amounts approximating that contemplated for in vivo use results in less than or equal to 20% cell death. A material is considered biocompatible with respect to a recipient if it is substantially nontoxic to the recipient's cells and tissues in the quantities and at the location used, and also does not elicit or cause a significant deleterious or untoward effect on the recipient's body, e.g., an immunological or inflammatory reaction, unacceptable scar tissue formation, etc.

“Biodegradable” means that a material is capable of being broken down physically and/or chemically within cells or within an extracellular compartment in the body of a subject, e.g., by hydrolysis under physiological conditions, by natural biological processes such as the action of enzymes present within cells or within the body, etc., to form smaller chemical species which can be metabolized and, optionally, reused, and/or excreted or otherwise disposed of. Materials that erode, distintegrate or deteriorate to smaller fragments, e.g., soluble molecules or supramolecular complexes, under physiological conditions are included within the scope of “biodegradable” materials. Preferably a biodegradable material is biocompatible.

A “biological macromolecule” is a large molecule composed of smaller subunits of a type that are found in biological systems. Examples of biological macromolecules include polypeptides, nucleic acids, and polysaccharides. Typically a biological macromolecule contains at least 3 subunits (e.g., amino acids, nucleosides, monosaccharides, etc.). The biological macromolecule may, but need not be, a naturally occurring polypeptide, nucleic acid, or polysaccharide. The biological macromolecule may be modified, e.g., it may be conjugated to a nonbiological molecule such as synthetic polymer, etc.

A “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, I, and properdin. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

A “complement regulatory protein” is a protein involved in regulating complement activity, such as the mammalian protein complement factor H (CFH).

A “complement control protein” is a complement regulatory protein comprising multiple SCR modules.

A “complement-like protein” is a protein that has significant sequence identity to a complement protein or a complement control protein over at least 20% of its length and/or specifically competes with the complement protein or complement control protein for binding to its target, e.g., has an affinity at least 10% as great. The genes encoding such proteins may be found in close proximity to genes encoding the complement protein or complement control protein having a similar sequence. For example, the CFH gene cluster contains numerous CFH-like genes (e.g., CFHR1, CFHR1, CFHR3, CFHR4, and CFHR5).

“Complement-related protein” refers collectively to complement components, complement regulatory proteins, and complement-like proteins; however, wherever the disclosure refers to complement-related proteins in general, it is understood that the invention encompasses embodiments that relate specifically to complement components, complement regulatory proteins, complement-like proteins, and combinations thereof.

An “effective amount” of an active agent such as a complement inhibitor refers to the amount of the active agent sufficient to elicit a desired biological response (or, equivalently, to inhibit an undesired biological response). As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, an effective amount may be an amount sufficient to relieve at least one symptom of a disorder. An effective amount may be an amount sufficient to slow the progression of a chronic and progressive disorder, e.g., to increase the time before one or more symptoms or signs of the disorder manifests itself or to increase the time before the individual suffering from the disorder reaches a certain level of impairment. An effective amount may be an amount sufficient to allow faster or greater recovery from an injury than would occur in the absence of the agent.

“Identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. By gap is meant a portion of a sequence that is not occupied by a residue. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between a sequence of interest and sequences in any of a variety of public databases. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. See the Web site having URL www.ncbi.nlm.nih.gov for these programs. In a specific embodiment, percent identity of a sequence of interest and a second sequence is calculated using BLAST2 with default parameters.

The term “isolated” means 1) separated from at least some of the components with which it is usually associated in nature; 2) prepared or purified by a process that involves the hand of man; and/or 3) not occurring in nature. For example, a molecule that is removed from a cell that produces it is “isolated”. A chemically synthesized molecule is “isolated”.

The term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

“Liquid composition” refers to a composition comprising at least one chemical substance that is a liquid at room temperature. A liquid composition may contain a therapeutic agent. The therapeutic agent may, for example, be dissolved, suspended, or dispersed therein. The therapeutic agent may be present as individual molecules interspersed with molecules of the liquid, or as microscopic or macroscopic aggregates or particles, etc., provided that such aggregates or particles do not prevent the composition from flowing readily, consistent with its being a liquid.

“Local administration” or “local delivery”, in reference to delivery of a composition or agent, refers to delivery that does not rely upon transport of the composition or agent to its intended target tissue or site via the vascular system. The composition or agent may be delivered directly to its intended target tissue or site, or in the vicinity thereof, e.g., in close proximity to the intended target tissue or site. For example, the composition may be delivered by injection or infusion of the composition. Following local administration in close proximity to a target tissue or site, the composition, or one or more components thereof, may diffuse to the intended target tissue or site. It will be understood that once having been locally delivered a fraction of a therapeutic agent (typically only a minor fraction of the administered dose) may enter the vascular system and be transported to another location, including back to its intended target tissue or site.

“Local activation” refers to complement activation that occurs outside the vascular system.

“Plurality” means more than one.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. Certain non-limiting suitable analogs and modifications are described in PCT publications WO2004026328 and WO2007062249. The polypeptide may be acetylated, e.g., at the N-terminus and/or amidated, e.g., at the C-terminus. The modifications can occur anywhere in a polypeptide, including the peptide backbone, the amino acid side-chains and the amino or carboxyl termini. A given polypeptide may contain many types of modifications. Polypeptides may be branched and/or cyclic, with or without branching. Polypeptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides (see, e.g., Kent, S., J Pept Sci., 9(9):574-93, 2003), or any combination of the foregoing. These methods are well known, and one of skill in the art will be able to select and implement an appropriate method for synthesizing the peptides and polypeptides described herein. A polypeptide may comprise one or more chemical ligation sites as described, for example, in U.S. Pub. No. 20040115774. In certain embodiments a polypeptide of the invention is modified with a polymer using one or more of the methods described or referenced therein. The term “polypeptide sequence” or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A polypeptide sequence presented herein is presented in an N-terminal to C-terminal direction unless otherwise indicated.

“Purified”, as used herein, means that an entity or substance is separated from one or more other entities or substances with which it was previously found before being purified. An entity or substance may be partially purified, substantially purified, or pure. A substance or entity such as a nucleic acid or polypeptide is considered pure when it is removed from substantially all other compounds or entities other than a solvent and any ions contained in the solvent, i.e., it constitutes at least about 90%, more preferably at least about 91%, 92%, 93%, 94%, 95%, 96%, 97%, 98%, 99%, or greater than 99% of the dry weight of the composition. A partially or substantially purified compound or entity such as a nucleic acid or polypeptide may be removed from at least 50%, at least 60%, at least 70%, or at least 80% by weight of the material with which it is naturally found, e.g., cellular material such as cellular proteins and/or nucleic acids. In certain embodiments the of a purified nucleic acid or polypeptide constitutes at least 10%, 20%, 30%, 40%, 50%, 60%, 70%, 80%, 90%, 95%, 99% or even more, by dry weight, of the total nucleic acid or polypeptide, respectively, in a composition. Methods for assessing purity are known in the art and include chromatographic methods, immunological methods, electrophoretic methods, etc. Any of the polynucleotides or polypeptides described herein may be purified.

“Reactive functional groups” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those frequently used to prepare bioconjugates, e.g., N-hydroxysuccinimide esters, maleimides, sulfhydryls, and the like (see, for example, Hermanson, G., Bioconjugate Techniques, Academic press, San Diego, 1996). Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “RNA interference” or “RNAi” is used herein as understood in the art, e.g., it refers to any method by which expression of a gene or gene product is decreased by introducing into, or expressing within, a target cell or organism a double-stranded RNAs (dsRNA), referred to as an “RNAi agent” which corresponds in sequence to the gene of interest, particularly short double-stranded RNAs (siRNAs) containing a strand that is complementary to messenger RNA of the gene of interest.

“Specific binding” generally refers to a physical association between a target polypeptide (or, more generally, a target molecule) and a binding molecule such as an antibody or ligand. The association is typically dependent upon the presence of a particular structural feature of the target such as an antigenic determinant, epitope, binding pocket or cleft, recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc.). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to detect and low affinity for nontarget molecules, the antibody will likely be an acceptable reagent. Once the specificity of a binding molecule is established in one or more contexts, it may be employed in other, preferably similar, contexts without necessarily re-evaluating its specificity. Binding of two or more molecules may be considered specific if the equilibrium dissociation constant (Kd) is 10⁻³ M or less, preferably 10⁻⁴ M or less, more preferably 10⁵ M or less, e.g., 10⁻⁶ M or less, 10⁻⁷ M or less, 10⁻⁸ M or less, or 10⁻⁹ M or less under the conditions tested, e.g., under physiological conditions.

“Significant sequence identity” as applied to an amino acid sequence means that the sequence is at least approximately 20%, 30%, 40%, 50%, 60%, 70%, 80%, 90%, or 95% identical to a reference sequence. In specific embodiments to an amino acid sequence means that the sequence is at least approximately 70%, 80%, 85%, 90%, 95%, 98%, or 99% identical to a reference sequence. In specific embodiments at least 20%, 30%, 40%, 50%, 60%, 70%, 80%, 90%, or 95% of the nonidentical amino acids are conservatively replaced relative to the reference sequence. Conservative replacements may be defined in accordance with Stryer, L., Biochemistry, 3rd ed., 1988, according to which amino acids in the following groups possess similar features with respect to side chain properties such as charge, hydrophobicity, aromaticity, etc. (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P, which may be considered to fall within group (1). In another accepted classification, conservative substitutions occur within the following groups: (1) Non-polar: A, L, I, V, G, P, F, W, M; (2) Polar: S, T, C, Y, N, Q. (3) Basic: K, R, H; (4) Acidic: D, E. Amino acids with a small side chain (G, A, S, T, M) also form a group from among which conservative substitutions can be made. Other classification methods known in the art can be used. Furthermore, amino acid analogs and unnatural amino acids can be classified in accordance with these schemes.

“Subject”, as used herein, refers to an individual to whom an agent is to be delivered, e.g., for experimental, diagnostic, and/or therapeutic purposes. Unless otherwise indicated, subjects are mammals, e.g., domesticated mammals such as dogs, cats, rabbits, etc., non-human primates, or humans.

“Systemic”, as used herein in reference to complement components, refers to complement proteins that are synthesized by liver hepatocytes and enter the bloodstream, or are synthesized by circulating macrophages or monocytes and secreted into the bloodstream.

“Therapeutic agent” is used herein to refer to any pharmacologically active agent useful for treating a disorder. The term includes any pharmaceutically acceptable salt, prodrug, salt of a prodrug, and such derivatives of such an agent as are known in the art or readily produced using standard methods known in the art. “Prodrug” refers to a precursor of an agent, wherein the prodrug is not itself pharmacologically active (or has a lesser or different activity than the desired activity of the drug) but is converted, following administration (e.g., by metabolism) into the pharmaceutically active drug. A therapeutic agent can be, without limitation, a small molecule or a biological macromolecule such as a polypeptide, antibody, or nucleic acid such as an aptamer, RNAi agent such as a small interfering RNA (siRNA), etc. A therapeutic agent is sometimes referred to as an “active agent” or “drug” herein. A small molecule is typically an organic compound having a molecular weight of 1,500 Daltons (Da) or less, e.g., 1,000 Da or less, e.g., 500 Da or less, and having multiple carbon-carbon bonds.

“Treating”, as used herein, refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of this invention can be administered to a subject who has developed a complement-mediated disorder or is at increased risk of developing such a disorder relative to a member of the general population. A composition of this invention can be administered prophylactically, i.e., before development of any symptom or manifestation of the condition (such as to a subject having at least one risk factor for developing the condition).

As used herein, “alkyl” refers to a saturated straight, branched, or cyclic hydrocarbon having from about 1 to about 22 carbon atoms (and all combinations and subcombinations of ranges and specific numbers of carbon atoms therein), with from about 1 to about 12, or about 1 to about 7 carbon atoms being preferred in certain embodiments of the invention. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinaations of ranges and specific number of carbon atoms) therein, e.g., from about 1 to 7 carbon atoms. Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like. “Lower alkanoyl” refers to an optionally substituted straight or branched aliphatic acyclic residue having about 1 to about 5 carbon atoms (and all combinations and subcombinaations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and have from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O-group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### DETAILED DESCRIPTION OF CERTAIN EMBODIMENTS OF THE INVENTION

Sustained Release of Compstatin Analogs From Gel-like Structures

The present invention relates to sustained delivery of a compstatin analog by release of the comptatin analog from a gel-like structure that forms upon introduction of a liquid composition comprising the compstatin analog into the body of a mammalian subject in an extravascular location such as the vitreous chamber. The invention also relates to sustained delivery of a compstatin analog by release of the comptatin analog from a gel-like structure that forms upon contacting a liquid composition comprising the compstatin analog with a body substance. The body substance is typically a fluid or fluid-containing substance other than blood (or substances derived therefrom such as plasma or serum) or urine. For example, the substance may be vitreous humor.

The body substance typically contains protein(s), inorganic or organic ion(s), and/or glycosaminoglycans. One or more of these constituents may initiate or contribute to formation of the gel-like structure. In certain embodiments of the invention the gel-like structure contains one or more endogenous proteins or glycosaminoglycans found in the body substance, wherein said endogenous protein(s) or glycosaminoglycan(s) are incorporated into the gel-like structure as it forms in vivo.

The invention also relates to sustained delivery of a therapeutic agent by introducing a liquid composition containing the therapeutic agent and the compstatin analog into an extravascular location in the body of a mammalian subject, whereby a gel-like structure is formed. The invention also relates to sustained delivery of a therapeutic agent by release of the therapeutic agent from a gel-like structure that forms upon contacting a liquid composition comprising a compstatin analog and the therapeutic agent with a body substance, e.g., a protein-containing body substance. The body substance is typically a fluid or fluid-containing substance other than blood (or fluids derived therefrom such as plasma or serum) or urine. For example, the substance may be vitreous humor.

The invention also relates to a gel-like structure formed outside the body (ex vivo) by contacting a liquid composition comprising a compstatin analog with one or more proteins, ions, or glycosaminoglycans, or a combination thereof, sufficient to drive gel formation. The proteins, ions, or glycosaminoglycans may be similar or essentially identical in structure or sequence to those naturally found in a body space such as the vitreous chamber, synovial cavity, etc. The gel-like structure may further contain an additional therapeutic agent. The invention further relates to sustained delivery of a compstatin analog and, optionally an additional therapeutic agent, by administering the gel-like structure to a subject in need thereof, e.g., by injecting, implanting, or applying it at a site where complement inhibition is desired.

In certain embodiments of the invention, the liquid composition is characterized in that it lacks gel-forming materials other than the compstatin analog. If the compstatin analog is omitted from the composition, no gel-like structure forms upon administering the liquid composition to a subject under circumstances in which a gel-like structure would readily form if the compstatin analog were present in suitable amounts, e.g., following intravitreal injection. In certain embodiments of the invention, the liquid composition is characterized in that it contains gel-forming materials, but such materials are not present in amounts sufficient to form a gel in the absence of the compstatin analog under circumstances in which a gel-like structure would readily form if the compstatin analog were present in suitable amounts, e.g., following intravitreal injection. In certain embodiments of the invention the liquid composition is thus distinct from compositions that contain gel-forming materials such as collagen, synthetic polymers, etc., which may be included in other compstatin analog formulations for the purpose of forming a gel in amounts sufficient to do so in the absence of a compstatin analog.

In certain embodiments of the invention the liquid composition consists essentially of a compstatin analog and one or more chemical substances that is a liquid at room temperature (e.g., water). In certain embodiments at least 50%, at least 60%, at least 70%, at least 80%, at least 85%, at least 90%, at least 95%, at least 97%, at least 98%, or at least 99% of the dry weight of the composition consists of the compstatin analog. In some embodiments the liquid composition contains water and an organic solvent. In some embodiments the liquid composition comprises, in addition to a compstatin analog, an inorganic ion, a buffer, a solubilizing agent, or a combination thereof, wherein such composition does not form a gel-like structure upon administration in the absence of the compstatin analog. Optionally, the liquid composition comprises a compstatin analog and one or more additional therapeutic agents.

The invention also relates to methods for modulating gel formation by a liquid composition comprising a compstatin analog and/or methods for modulating properties of a gel formed from a liquid composition comprising a compstatin analog, and to methods of testing agents to determine whether they desirably modulate one or more properties of the gel and/or one or more characteristics of formation (e.g., rate at which the gel forms or disappears, density, consistency, compstatin analog content, rate of release of compstatin analog, etc.). The methods include preparing a liquid composition comprising a compstatin analog, a liquid substance (e.g., water), and a test agent; administering the liquid composition to an extravascular location of a mammalian subject (e.g., the vitreous chamber); and assessing one or more properties of the composition in vivo following administration. The method is used to identify agents that desirably affect one or more properties of the gel and/or its formation. Agents to be tested include standard excipients, viscosity modulators, inorganic and organic ions, buffers, etc.

One aspect of the invention is liquid compositions comprising a compstatin analog, a liquid substance, and a modulator of one or more properties of the gel or of gel formation.

Compstatin is a cyclic peptide that binds to complement component C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 44), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), U.S. Ser. No. 11/544,389 (Compstatin and Analogs Thereof for Eye Disorders) and discussion below.

The present invention arose at least in part as a result of the unexpected discovery that intravitreal administration of a liquid composition containing a potent compstatin analog, in water results in formation of a macroscopic, gel-like, intravitreal structure, also referred to as a “deposit” or simply as a “gel” (see FIG. 1). “Gel-like” as used herein refers to a gel or to any composition of matter having physical properties characteristic of a gel (e.g., gelatin). The term “gel-like” is thus intended to describe the physical nature of the jelly-like or gelatinous structure rather than to represent a particular physical structure. Exemplary physical properties are viscosity, elasticity, hardness, and compressibility. The term “gel-like” includes but is not limited to compositions which may have the same homogeneous structure as is generally attributed to gels by those skilled in the art of colloid chemistry, wherein a gel may be defined as colloidal system in which a porous network of interconnected particles (typically of nanometer scale) spans the volume of a liquid medium. The extravascular location may contain a body substance, which may have varying degrees of viscosity. The substance may be a body fluid. The substance may itself be at least somewhat gel-like in consistency. In certain embodiments of the present invention the gel-like structure formed following administration of the compstatin analog has a greater viscosity and more solid consistency than the contents of the extravascular location in which it forms.

The inventors recognized that the deposit could serve as a unique sustained delivery system for the compstatin analog. It was discovered that the deposit contains a significant fraction of the administered compstatin analog and that the compound retains activity and is released over time. The deposit is a discrete structure that can frequently be separated from the vitreous material upon dissection shortly after it is formed and when a sufficiently high concentration of compstatin analog is used. With time and/or if lower concentrations are used, the gels are not very “solid” and are soft and easily broken apart upon manipulation, consistent with their disintegration over time. In certain embodiments of the invention the gels are approximately spheroidal and substantially translucent. In certain embodiments the macroscopic gel-like structure diminishes in size and/or density (as observed using sonography) over time and releases the compstatin analog in active form so as to achieve a therapeutic concentration for at least 2 weeks, at least 4 weeks, at least 2 months, at least 3 months, at least 6 months, at least 9 months, or at least 12 months (e.g., up to about 3, 6, 9, 12, 18, or 24 months in different embodiments). The extravascular location may be a discrete chamber, compartment, or cavity, which may contain a fluid or semi-fluid body substance. The location may be the vitreous chamber, a synovial cavity, the intrathecal space, etc. The chamber, cavity, or compartment may be at least in part lined with, or may contain, a tissue on which complement exerts undesirable effects in a complement-mediated disorder. In some embodiments, the tissue does not directly contact the interior of the space but is sufficiently close to it that the complement inhibitor can diffuse into the extracellular fluid bathing the tissue. For example, the tissue may be nearby, e.g., located within 10-20 mm, in some embodiments within 5 mm-10 mm, within 1 mm-5 mm, within 0.5 mm-1 mm, within 0.1 mm-0.5 mm, or within less than 0.1 mm from the lining of the compartment, and not separated from it by a barrier that would prevent diffusion of the complement inhibitor to the tissue. The therapeutic concentration may be measured within a substance found within the extravascular location (e.g., vitreous humor) or within tissues lining it, or nearby tissues.

In certain embodiments the macroscopic gel-like structure remains readily detectable for at least 2 weeks, at least 4 weeks, at least 2 months, at least 3 months, at least 6 months, at least 9 months, or at least 12 months (e.g., up to about 3, 6, 9, 12, 18, or 24 months in different embodiments). It will be appreciated that there may be variability between subjects, and the afore-mentioned values may reflect averages among a population of subjects studied. “Readily detectable” means that the deposit can be seen (i) upon standard ophthalmoscopic examination; (ii) without magnification upon dissection and/or (iii) using ultrasound imaging. In some embodiments the deposit has an initial diameter (or longest axial dimension or greatest distance between two points on the surface) of about 5 mm but can be smaller or larger depending on, e.g., the volume of liquid composition administered and the concentration of compstatin analog. In some embodiments, the macroscopic gel-like structure remains at least 1 mm in diameter (or longest axial dimension or greatest distance between two points on the surface) for at least 2 weeks, at least 4 weeks, at least 2 months, at least 3 months, at least 6 months, at least 9 months, or at least 12 months (e.g., up to about 3, 6, 9, 12, 18, or 24 months in different embodiments).

Typically, only a fraction of the administered compstatin analog is captured within the deposit. As the concentration and total amount of the compound injected increase, the fraction of the administered dose that is subsequently found in the deposit increases. In certain embodiments of the invention, at least 25% of the administered dose is initially retained in the deposit. For example, in certain embodiments between 25% and 50%, or between 50% and 75% of the administered compstatin analog is retained in the deposit. In certain embodiments of the invention, between 75% and 90% of the administered compstatin analog is initially retained in the deposit. It will be appreciated that there may be variability between subjects, and the afore-mentioned values may reflect averages among a population of subjects studied.

The deposit is apparently non-toxic at the doses tested and does not appear to alter the normal physiology of the eye or to interfere with vision. It contains, in addition to the compstatin analog, proteins normally present in the vitreous. The deposit persists over an extended period of time, during which it releases the compstatin analog. “Release” as used herein refers generally to making the active agent (e.g., compstatin analog) available outside the deposit (e.g., within the body) but does not imply any particular mechanism by which the process occurs. Release may, for example, occur as the deposit degrades or disintegrates, by diffusion of the agent out of the deposit, etc. The released compstatin analog retains substantial activity. In some embodiments, on a molar basis, the released compstatin analog retains at least 50% of the activity of the administered compound (e.g., between 50% and 75%, between 75% and 95%, between 95% and 100% of the original activity.

The deposit thus provides a depot of the compstatin analog and affords a novel means for sustained delivery. It is contemplated that gel-like deposits resembling those formed following intravitreal administration may form in vivo when a liquid composition comprising a compstatin analog is introduced into extravascular locations containing body fluids such as those present in the synovial cavity, bursae, the cranial cavity, ventricles in the cranial cavity, intrathecal space, etc. Such deposits would contain the compstatin analog and one or more substances such as ions, proteins, or glycosaminoglycans present in the respective body fluid found in such location. It is also contemplated that gel-like deposits may form ex vivo from a liquid composition comprising a compstatin analog and one or more proteins, ions, or glycosaminoglycans, similar or identical to those found in vivo in an extravascular location.

The invention further provides a method of treating a complement-mediated disorder comprising the step of: administering a liquid composition comprising an effective amount of a compstatin analog to an extravascular location of a subject, wherein said effective amount is sufficient to form a discrete, macroscopic gel-like structure containing the compstatin analog within said extravascular location. In certain embodiments of the invention the composition is administered into the vitreous chamber, e.g., by intravitreal injection. In certain embodiments a 27 gauge-30 gauge needle is used.

The invention further provides a gel-like material comprising a compstatin analog and one or more proteins, ions, and/or glycosaminoglycans (GAGs) naturally found in an extravascular location of a mammalian subject. In one embodiment the proteins, ions, and/or glycosaminoglycans are normally found in the vitreous humor. In another embodiment the proteins, ions, and/or glycosaminoglycans are normally found in synovial fluid. In another embodiment the proteins, ions, and/or glycosaminoglycans are normally found in cerebrospinal fluid (CSF). In certain embodiments of the invention the gel-like material contains at least 2, 3, 4, 5, 6, 7, 8, 9, 10, or more distinct proteins and/or GAGs. In certain embodiments of the invention the protein or GAG is not a substance that is conventionally used in the art to make sustained release compositions. In certain embodiments of the invention the protein or GAG is not a therapeutic agent known in the art.

In certain embodiments of the invention, the gel-like material is present in or isolated from a subject. In these embodiments, the gel-like material may contain proteins, ions, and/or glycosaminoglycans that are endogenous to that particular subject (i.e., they are naturally found in that particular subject). In certain embodiments the gel-like material is formed ex vivo. In the latter case, the deposit may contain proteins, ions, or glycosaminoglycans that are similar or identical to those in a subject to which the gel is to be administered, but are not obtained from that subject. For example, they could be purified from natural sources, synthesized using chemical or recombinant synthesis techniques, etc.

In certain embodiments of the invention the compstatin analog is administered under conditions appropriate to form a gel-like deposit in the vitreous chamber in order to treat a subject at risk of or suffering from a disorder affecting the eye. In some embodiments of the invention the disorder is one in which increased complement activation (e.g., as evidenced by increased levels of complement activation products) is detectable in the eye in subjects suffering from the disorder as compared with complement activation in the eyes of individuals not suffering from the disorder. In some embodiments of the invention the disorder is one in which an increased MAC level is detectable in the eye in subjects suffering from the disorder as compared with MAC levels in the eyes of individuals not suffering from the disorder.

In some embodiments the disorder is macular degeneration, e.g., age-related macular degeneration (AMD). In some embodiments the disorder is wet type AMD. In some embodiments the disorder is dry type AMD. In some embodiments the subject suffers from geographic atrophy (GA). In some embodiments the subject suffers from age-related maculopathy, which term refers to early to intermediate dry AMD in which GA has not developed. In some embodiments the disorder is diabetic retinopathy. In some embodiments the disorder is glaucoma. In some embodiments the eye disorder is posterior uveitis or keratitis. In some embodiments the eye disorder is retinitis pigmentosa. Further information about these and other eye disorders is found in copending patent applications U.S. Ser. No. 11/247,886, U.S. Ser. No. 11/544,389, and U.S. Ser. No. 11/612,751 and in Kanski, J., Clinical Ophthalmology: A Systematic Approach Butterworth-Heinemann; 6 edition (May 4, 2007). In some embodiments the composition is administered intravitreally, e.g., by intravitreal injection. In some embodiments the composition is administered to the anterior chamber of the eye.

The extravascular location is selected as appropriate for the disorder being treated. For example, if the condition is arthritis the complement inhibitor may be administered directly into a synovial cavity. Examples of intra-articular joints where the liquid compositions of the invention can be administered include hip, knee, elbow, wrist, sternoclavicular, temperomandibular, carpal, tarsal, ankle, and any other joint subject to arthritic conditions. The compositions are also suitable for administration to bursae. Examples of bursae to which the compositions of the invention can be administered include acromial, bicipitoradial, cubitoradial, deltoid, infrapatellar, ischial, and other bursae known to those skilled in the art. If the condition is spinal cord injury or chronic pain the composition may be administered intrathecally. If the condition is stroke, Alzheimer's disease, Parkinson's disease, or stroke, the composition may be administered into the ventricular system (e.g., third, fourth, or lateral ventricles), which comprises the set of structures in the brain continuous with the central canal of the spinal cord. Methods known in the art for administering therapeutic agents into these locations may be used (see below).

Suitable amounts of the compstatin analog can be selected at least in part based on the level of complement proteins and/or complement activation within the contents of an extravascular location of interest or nearby tissues. The determination could be made on the basis of measurements made in multiple subjects in an individual subject to be treated. In certain embodiments of the invention an appropriate dosage of a compstatin analog is selected based at least in part on this information and, optionally by estimating a total amount of complement protein using the approximate volume of the space. For example, an appropriate dose of a compstatin analog may be selected to achieve an average or steady state concentration in the space at least sufficient to bind to 50%, 60%, 70%, 80%, 90%, 95%, or more of the C3 present. In certain embodiments the dose is selected to achieve an average or steady state concentration equal to 1, 2, 5, 10, 20, 50, or any intervening subrange between 1 and 50, times as great as that of C3. In certain embodiments of the invention the compstatin analog is released from the deposit so as to provide an effective average or steady state concentration for a prolonged period of time, e.g., 2-4 weeks, 4-6 weeks, 1-3 months, 3-6 months, 6-12 months, 12-24 months. In some embodiments the dose is selected to achieve a release rate in the vitreous chamber of between about 0.01 μg/day and about 3-5 μg/day for, e.g., between about 0.05 μg/day and about 3 μg/day, between 0.1 μg/day and about 3 μg/day, about 0.5 μg/day and about 3 μg/day, about 0.5 μg/day and 2 μg/day, etc., for at least 2 weeks, e.g., 2-4 weeks, 4-6 weeks, 1-3 months, 3-6 months, 6-12 months, 12-24 months. In some embodiments the dose is selected to achieve a release rate in the vitreous chamber of between about 0.5 μg/day and 1 μg/day or between about 1 μg/day and about 3 μg/day for at least 2 weeks, e.g., 2-4 weeks, 4-6 weeks, 1-3 months, 3-6 months, 6-12 months, 12-24 months.

The invention provides a unit dosage of the compositions described herein comprising, typically in a container, a sufficient amount of the liquid composition (or a dry powder that can be combined with a liquid pharmaceutical carrier to yield a liquid composition) to produce a desired therapeutic effect in a patient, i.e., a sufficient amount for a single administration to a patient in need thereof. In one embodiment, the unit dosage is sterile and lyophilized. In another embodiment the unit dosage is sterile and provided as a liquid acceptable for administration to a patient, e.g., by injection or infiltration. In another embodiment the unit dosage is a suspension or dispersion in a liquid suitable for administration to a patient, e.g., by injection, infiltration, etc. In some embodiments the liquid composition contains at least 1 mg compstatin analog per ml liquid, e.g., between 1 mg and about 300 mg compstatin analog per ml liquid. The compstatin analog need not be completely dissolved in the liquid, e.g., at least a portion of the compstatin analog may be present in particulate form. In some embodiments the concentration of compstatin analog in the composition is between 2 mg/ml and 20 mg/ml. In some embodiments the concentration of compstatin analog in the composition is between 2 mg/ml and 10 mg/ml, e.g., between 2 mg/ml and 5 mg/ml. In some embodiments the concentration of compstatin analog in the composition is between 8 mg/ml and 25 mg/ml In some embodiments the concentration of compstatin analog dissolved is between 2 mg/ml and 20 mg/ml. In some embodiments the concentration of compstatin analog dissolved is between 2 mg/ml and 10 mg/ml, e.g., between 2 mg/ml and 5 mg/ml. In some embodiments the concentration of compstatin analog dissolved is between 8 mg/ml and 25 mg/ml.

In some embodiments the compstatin analog is administered to the vitreous chamber (e.g., by intravitreal injection) at a concentration of at least 1 mg/ml, e.g., between 1 mg/ml and 10 mg/ml, e.g., between 2 mg/ml and 5 mg/ml in a volume of at least 25 μl. In some embodiments the volume is between 25 μl and 150 μl, e.g., between 40 μl and 125 μl, e.g., between 50 μl and 100 μl. In some embodiments the volume is about 50 μl, e.g., between 45 μl and 55 μl. In some embodiments the volume is about 100 μl, e.g., between 90 μl and 110 μl. In some embodiments between 50 μg and 2 mg compstatin analog is administered, e.g., between 100 μg and 500 μg. In some embodiments between 500 μg and 1 mg compstatin analog is administered. In some embodiments between 1 mg and 1.5 mg compstatin analog is administered. In some embodiments between 1.5 mg and 2.0 mg compstatin analog is administered.

In some embodiments, between about 150 μg and about 250 μg compstatin analog is administered to the vitreous chamber. In some embodiments, between about 250 μg and about 350 μg compstatin analog is administered to the vitreous chamber. In some embodiments, between about 350 μg and about 450 μg compstatin analog is administered to the vitreous chamber. In some embodiments, between about 450 μg and about 550 μg compstatin analog is administered to the vitreous chamber. In some embodiments, between about 550 μg and about 650 μg compstatin analog is administered to the vitreous chamber. In some embodiments, between about 650 μg and about 750 μg compstatin analog is administered to the vitreous chamber. In some embodiments, between about 750 μg and about 850 μg compstatin analog is administered to the vitreous chamber. In some embodiments, between about 850 μg and about 950 μg compstatin analog is administered to the vitreous chamber. In some embodiments, between about 950 μg and about 1050 μg compstatin analog is administered to the vitreous chamber. In some embodiments, between about 1050 μg and about 1150 μg compstatin analog is administered to the vitreous chamber. In some embodiments, between about 1150 μg and about 1250 μg compstatin analog is administered to the vitreous chamber. In some embodiments, between about 1250 μg and about 1350 μg compstatin analog is administered to the vitreous chamber. In some embodiments, between about 1350 μg and about 1450 μg compstatin analog is administered to the vitreous chamber. In some embodiments, between about 1450 μg and about 1550 μg compstatin analog is administered to the vitreous chamber. In some embodiments, between about 1550 μg and about 1650 μg compstatin analog is administered to the vitreous chamber. In some embodiments, between about 1650 μg and about 1750 μg compstatin analog is administered to the vitreous chamber. In some embodiments, between about 1850 μg and about 1950 μg compstatin analog is administered to the vitreous chamber. In some embodiments, between about 1950 μg and about 2050 μg compstatin analog is administered to the vitreous chamber.

As described in Example 3 and shown in FIG. 5, experiments showed that a compstatin analog can be measured in the serum after intravitreal administration. The serum concentration was found to correlate well with the vitreal concentration. The invention provides a method of assessing the local concentration of a compstatin analog after local administration to an extravascular location, the method comprising: measuring the concentration of the compstatin analog in the blood following administration of the compstatin analog to the extravascular location and determining the local concentration in said location based on a known correlation between the concentration in the blood and the concentration in said location. The invention provides a method of assessing the vitreal concentration of a compstatin analog comprising: measuring the concentration of the compstatin analog in the blood following intraocular (e.g., intravitreal) administration of the compstatin analog and determining the vitreal concentration based on a known correlation between the concentration in the blood and the vitreal concentration. These methods afford a convenient way to monitor the local concentration (e.g., vitreal concentration) of the compstatin analog and provides a means to determine when the patient should be retreated. For example, when the serum concentration (and hence the local, e.g., vitreal, concentration) falls below a desired value the patient is retreated. Hence this method permits the practitioner to tailor the interval between doses to the particular patient being treated.

The invention thus provides a method of monitoring a subject following local administration (e.g., intraocular administration) of a compstatin analog. It will be appreciated that the assay may be performed on plasma or serum using a variety of methods, e.g., using a high performance liquid chromatography (HPLC) assay using either ultraviolet or fluorescence detection along or in conjunction with mass spectrometry (LC-MS).

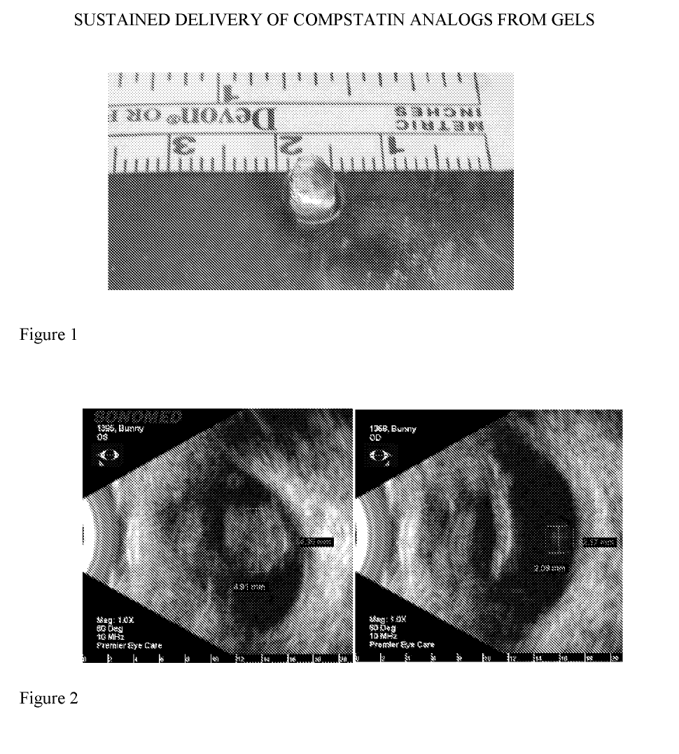

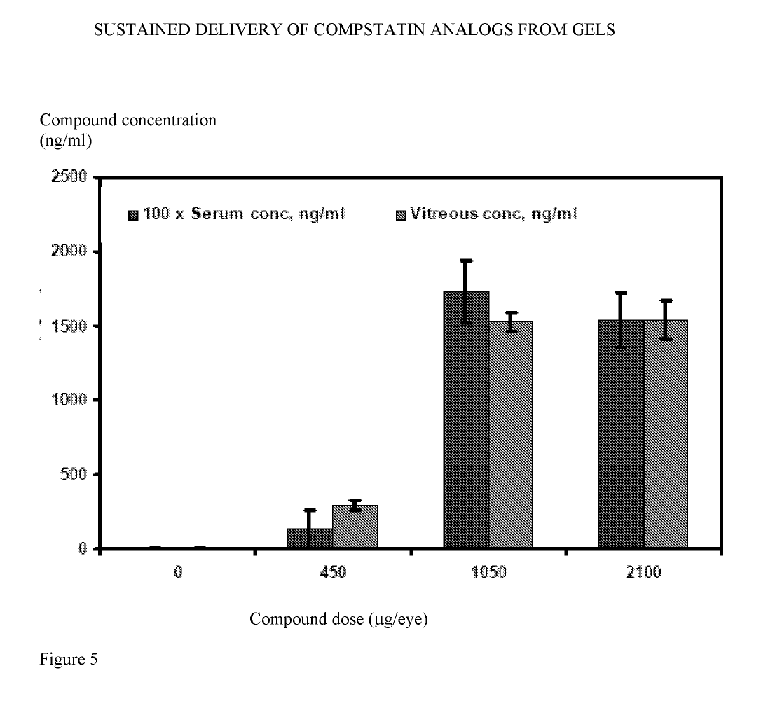

Furthermore, it was discovered that the gel-like structure is detectable using ultrasound (FIG. 2). The size and density of the deposit can be monitored over time, thereby providing a second means of monitoring the subject and determining when to retreat. For example, retreatment can be provided when the diameter or density of the deposit falls below a preselected threshold. The serum assay can be used together with ultrasound or without the use of ultrasound to monitor the size of the deposit and provide information relevant to the decision of whether and when to retreat.

It will be understood that a compstatin analog may form a gel-like deposit in some subjects, but not necessarily all subjects, at a given dose. It will also be understood that a compstatin analog may form a gel-like deposit when introduced into some extravascular locations but not others. The dose and specific compstatin analog are selected as appropriate. For example, a dose and compstatin analog can be selected that has been shown to form a deposit in at least 50%, at least 60%, at least 70%, at least 80%, at least 90%, or at least 95% of subjects (e.g., non-human primates, or humans) when administered to a particular extravascular location.

Compstatin and Analogs Thereof

As described above, the invention arose at least in part as a result of the serendipitous and unexpected discovery that liquid compositions containing a sufficient quantity and/or concentration of a potent compstatin analog form macroscopic, gel-like structures when administered to the vitreous chamber of a mammalian subject. While not wishing to be bound by any theory, the inventors propose that this property may be exhibited by a variety of compstatin analogs. Based on the disclosure herein, the skilled artisan can readily test compstatin analogs to determine whether they exhibit similar properties when introduced into the vitreous chamber or into a different extravascular location. Liquid compositions comprising such compstatin analogs and methods of using such compositions to administer the compstatin analog and, optionally, an additional active agent, are aspects of the invention. In certain embodiments the liquid composition comprises first and second compstatin analogs, wherein at least one of the compstatin analogs forms a gel-like deposit upon administration to an extravascular location. In certain embodiments the liquid composition comprises a compstatin analog and a second active agent that is not a compstatin analog.

As used herein, the term “compstatin analog” encompasses compstatin and any complement inhibiting analog thereof. It should be understood where the term “compstatin analog” is used in the instant application to describe or refer to an aspect of the invention, the invention includes an embodiment in which the compstatin analog is the compound studied as described in the Examples (SEQ ID NO: 32), unless otherwise indicated. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. The assay may, for example, measure classical or alternative pathway-mediated erythrocyte lysis. In certain embodiments the assay is an ELISA assay. WO2004/026328, Morikis, supra, Mallik, supra, Katragadda 2006, supra, among other references, describe methods for determining the ability of a compound to inhibit complement activation. Concatamers or multimers of compstatin or a complement inhibiting analog (with appropriate modification of the N- and/or C-termini) thereof are also of use in the present invention.

The activity of a compstatin analog may be expressed in terms of its IC₅₀ (the concentration of the compound that inhibits complement activation by 50%), e.g., at a particular plasma concentration, with a lower IC₅₀ indicating a higher activity as recognized in the art. The activity of a preferred compstatin analog for use in the present invention is at least as great as that of compstatin. Certain modifications are known to reduce or eliminate complement inhibiting activity and may be explicitly excluded from any embodiment of the invention. It will be appreciated that the precise IC₅₀ value measured for a given compstatin analog will vary with experimental conditions. Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments of the invention the activity of the compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments of the invention the compstatin analog is a potent compstatin analog such as an analog having an activity at least 100-fold greater than that of compstatin. In certain embodiments of the invention the activity of the compstatin analog is at least 150, at least 200, or at least 250 times as great as that of compstatin. Table 1 presents sequences of a number of such analogs. In certain embodiments the invention contemplates use of analogs whose activity is between 264 and 300, 300 and 350, 350 and 400, or 400 and 500 times as great as that of compstatin. The invention further contemplates use of compstatin analogs having activities between 500 and 1000 times that of compstatin.

The K_{d} of binding to C3 has been measured for various compstatin analogsusing isothermal titration calorimetry (Katragadda, et al., J. Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μM and 0.1 μM, between 0.025 μM and 0.05 μM, between 0.015 μM and 0.025 μM, between 0.01 μM and 0.015 μM, or between 0.001 μM and 0.01 μM. In certain embodiments the IC₅₀ of the compstatin analog is between about 0.2 μM and about 0.5 μM. In certain embodiments the IC₅₀ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH-W), 5-hydroxy-L-tryptophan (5OH-W), 6-hydroxy-L-tryptophan (6OH-W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (αMeW), β-methyl-L-tryptophan (βMeW), N-methyl-L-tryptophan (NMeW), 5-methoxy-tryptophan, ornithine (orn), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring. Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components.

In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine In certain embodiments of the invention X′aa is an analog of Trp that has increased hydrophobic character relative to Trp. For example, X′aa may be 1-methyltryptophan. In certain embodiments of the invention Xaa is an analog of Trp that has increased propensity to form hydrogen bonds relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa -Gly-X″aa (SEQ ID NO: 4), where X′aa and Xaa are each independently selected from Trp and analogs of Trp and X″aa is selected from His, Ala, analogs of Ala, Phe, and Trp. In certain embodiments of the invention X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine In certain embodiments of the invention Xaa is an analog of Trp that has increased propensity to form hydrogen bonds with C3 relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments X″aa is selected from analogs of Trp, aromatic amino acids, and aromatic amino acid analogs.

In certain preferred embodiments of the invention the compstatin analog is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10-12 amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S—S) bond. In other embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:
Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe or an analog of Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond.

In other embodiments Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-Nal), 1-naphthylalanine (1-Nal), 2-indanylglycine carboxylic acid (Ig1), dihydrotrpytophan (Dht), and 4-benzoyl-L-phenylalanine

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

In certain embodiments the invention Xaa3 is Phe.

In certain embodiments the invention employs a compstatin analog of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or vitreous. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group), also referred to herein as “alkanoyl”. The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the negative charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:
Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe or an analog of Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and
the two Cys residues are joined by a disulfide bond.

Xaa1, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments of the invention Xaa1 is Ile and Xaa4 is L-Thr.

In certain embodiments of the invention Xaa1 is Ile, Xaa2* is Trp, and Xaa4 is L-Thr.

In certain embodiments the invention utilizes a compstatin analog of SEQ ID NO: 7, as described above, wherein Xaa2 and Xaa2* are independently selected from Tip, analogs of Trp, other amino acids or aromatic amino acid analogs, and Xaa3 is His, Ala or an analog of Ala, Phe or an analog of Phe, Trp, an analog of Trp, or another aromatic amino acid or aromatic amino acid analog.

In certain embodiments of any of the compstatin analogs described herein, Xaa3 is an analog of His. In certain embodiments of any of the compstatin analogs described herein Xaa3 is Phe.

Table 1 provides a non-limiting list of compstatin analogs useful in various embodiments of the present invention. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides are amidated at the C-terminus. Bold text is used to indicate certain modifications. Activity relative to compstatin is based on published data and assays described therein (e.g., WO2004/026326, Mallik, 2005; Katragadda, 2006; WO2007/062249). Where multiple publications reporting an activity were consulted, the more recently published value is used, and it will be recognized that values may be adjusted in the case of differences between assays. It will also be appreciated that the peptides listed in Table 1 are typically cyclized via a disulfide bond between the two Cys residues when used in the compositions and methods of the invention. However, other means of cyclizing the peptides may be used.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3× more |
| AC-V4Y/H9A | Ac-ICVYQDWGAHRCT-CONH2 | 10 | 14× more |
| Ac-V4W/H9A-OH | Ac-ICVWQDWGAHRCT-COOH | 11 | 27× more |
| AC-V4W/H9A | Ac-lCVWQDWGAHRCT-CONH2 | 12 | 45× more |
| Ac-V4W/H9A/T13dT-OH | Ac-ICVWQDWGAHRCdT-COOH | 13 | 55× more |
| Ac-V4(2-Nal)/H9A | Ac-ICV(2-Nal)QDWGAHRCT-CONH2 | 14 | 99× more |
| Ac-V4(2-Nal)/H9A-OH | Ac-ICV(2-Nal)QDWGAHRCT-COOH | 15 | 38× more |
| Ac-V4(1-Nal)/H9A-OH | Ac-ICV(1-Nal)QDWGAHRCT-COOH | 16 | 30× more |
| Ac-V42Igl/H9A | Ac-ICV(2-Igl)GDWGAHRCT-CONH2 | 17 | 39× more |
| Ac-V42Igl/H9A-OH | Ac-ICV(2-Igl}QDWGAHRCT-COOH | 18 | 37× more |
| Ac-V4Dht/H9A-OH | Ac-ICVDhtQDWGAHRCT-COOH | 19 | 5× more |
| Ac-V4(Bpa)/H9A-OH | Ac-ICV(Bpa)QDWGAHRCT-COOH | 20 | 49× more |
| Ac-V4(Bpa)/H9A | Ac-ICV(Bpa)QDWGAHRCT-CONH2 | 21 | 86× more |
| Ac-V4(Bta)/H9A-OH | Ac-ICV(Bta)QDWGAHRCT-COOH | 22 | 65× more |
| Ac-V4(Bta)/H9A | Ac-ICV(Bta)QDWGAHRCT-CONH2 | 23 | 64× more |
| Ac-V4W/H9(2-Abu) | Ac-ICVWQDWG(2-Abu)HRCT-CONH2 | 24 | 64× more |
| +G/V4W/H9A + AN-OH | H-GICVWQDWGAHRCTAN-COOH | 25 | 38× more |
| Ac-V4(5fW)/H9A | Ac-ICV(5fW)QDWGAHRCT-CONH2 | 26 | 31× more |
| Ac-V4(5-MeW)/H9A | Ac-ICV(5-methyl-W)QDWGAHRCT-CONH2 | 27 | 67× more |
| Ac-V4(1MeW)/W7(5fW)/H9A | Ac-ICV(1-methyl-W)QD(5fW)GAHRCT-CONH2 | 28 | 264× more |
| Ac-V4W/W7(5fW)/H9A | Ac-ICVWQD(5fW)GAHRCT-CONH2 | 29 | 121× more |
| Ac-V4(5fW)/W7(5fW)/H9A | Ac-ICV(5fW)QD(5fW)GAHRCT-CONH2 | 30 | 161× more |
| Ac-V4(5-MeW)/W7(5fW)H9A | Ac-ICV(5-methyl-W)QD(5fW)GAHRCT-CONH2 | 31 | NA |
| Ac-V4(1-MeW)/H9A | Ac-ICV(1-methyl-W)QDWGAHRCT-CONH2 | 32 | 264× more |
| +G/V4(6fW)/W7(6fW)H9A + N—OH | H-GICV(6fW)QD(6fW)GAHRCTN-COOH | 33 | 126× more |
| Ac-V4(1-formyl-W)/H9A | Ac-ICV(1-formyl-W)QDWGAHRCT-CONH2 | 34 | 264× more |
| Ac-V4(5-methoxy-W)/H9A | Ac-ICV(1-methyoxy-W)QDWGAHRCT-CONH2 | 35 | 76× more |
| G/V4(5f-W)/W7(5fW)/H9A + N—OH | H-GICV(5fW)QD(5fW)GAHRCTN-COOH | 36 | 112× more |

NA = not available

In certain embodiments of the compositions and methods of the invention the compstatin analog has a sequence selected from sequences 9-36. In certain embodiments of the compositions and methods of the invention the compstatin analog has a sequence selected from SEQ ID NOs: 14, 21, 28, 29, 32, 33, 34, and 36. In certain embodiments of the compositions and methods of the invention the compstatin analog has SEQ ID NO: 28. In certain embodiments of the compositions and methods of the invention the compstatin analog has SEQ ID NO: 29. In certain embodiments of the compositions and methods of the invention the compstatin analog has a sequence selected from SEQ ID NOs: 30 and 31. In certain embodiments of the compositions and methods of the invention the compstatin analog has SEQ ID NO: 32. In certain embodiments of the compositions and methods of the invention the compstatin analog has SEQ ID NO: 33. In certain embodiments of the compositions and methods of the invention the compstatin analog has SEQ ID NO: 34. In certain embodiments of the compositions and methods of the invention the compstatin analog has SEQ ID NO: 36.

In other embodiments, compstatin analogs having sequences as set forth in Table 1, but where the Ac-group is replaced by an alternate blocking moiety B¹, as described above, are used. In other embodiments, compstatin analogs having sequences as set forth in Table 1, but where the —NH₂ group is replaced by an alternate blocking moiety B², as described above, are used.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 1, e.g., SEQ ID NO: 14, 21, 28, 29, or 32 in a peptide-C3 structure, e.g., a crystal structure. In certain embodiments the compstatin analog is a compound that binds to the binding site of a peptide having SEQ ID NO: 30 or 31 in a peptide-C3 structure, e.g., a crystal structure. In certain embodiments the compstatin analog is a compound that could substitute for the peptide of SEQ ID NO: 9-36, e.g., SEQ ID NO: 14, 21, 28, 29, 30,32, 34, or 36 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof. One of skill in the art would be able to use molecular modeling software programs to predict whether a compound would form substantially the same intermolecular contacts with C3 as would compstatin or a peptide having the sequence of one or more of the peptides in Table 1, e.g., SEQ ID NO: 14, 21, 28, 29, or 32, or in other embodiments SEQ ID NO: 30 or 31.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, and/or WO2004026328. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

The structure of compstatin is known in the art, and NMR structures for a number of compstatin analogs having higher activity than compstatin are also known (Malik, supra). Structural information may be used to design compstatin mimetics. In one embodiment, the compstatin mimetic is any compound that competes with compstatin or any compstatin analog (e.g., a compstatin analog whose sequence is set forth in Table 1) for binding to C3 or a fragment thereof (such as a 40 kD fragment of the β chain to which compstatin binds) and that has an activity equal to or greater than that of compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In embodiments the compstatin mimetic is a compound that binds to the binding site of a peptide having a sequence set forth in Table 1 in a peptide-C3 structure. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455), Eguchi M, Kahn M., Mini Rev Med Chem., 2(5):447-62, 2002. The design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques. Virtual screening methods can be used to identify compstatin mimetics that bind to C3. Such methods may comprise use of suitable algorithms to computationally dock, score, and optionally rank a plurality of candidate structures. Any of a wide variety of available software programs can be used to perform the virtual screening method. Exemplary programs useful for flexible molecular docking include DOCK 4.0, FlexX 1.8, AutoDock 3.0, GOLD 1.2, ICM 2.8, and more recent versions thereof.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Combination Therapies and Compositions

The invention provides liquid compositions comprising: (a) a compstatin analog in an amount sufficient to form a macroscopic, gel-like structure upon introduction into an extravascular location of a mammalian subject; and (b) an additional therapeutic agent, wherein said additional therapeutic agent is not a compstatin analog. The present invention contemplates the use of compstatin analogs together with one or more other second agents effective for treatment of a disorder. The agents may act on the same target(s) or pathway(s) or on different targets or pathways. The compstatin analog and the second agent may act additively or synergistically (wherein the combined activity of the agents is greater than the sum of their activities if administered individually). In some embodiments the second agent is administered to treat a disorder not associated with complement activation, i.e., the gel-like structure is used essentially as a sustained release delivery system for the second agent. In some embodiments, a peptide comprising a sequence selected from SEQ ID NOs: 3, 4, 5, 6, or 7 is used to form a gel-like structure, wherein the peptide has lower complement inhibiting activity than compstatin. In some embodiments, the peptide has 50% or less activity than compstatin.

The second agent(s) are selected as appropriate for the disorder to be treated. Suitable agents include anti-inflammatory agents such as corticosteroids, non-steroidal anti-inflammatory agents, leukotriene or leukotriene receptor antagonists, cytokine or cytokine receptor antagonists (e.g., anti-TNFα agents such as antibodies or soluble TNFα receptors or fragments thereof that bind TNFα), anti-IgE agents (e.g. antibodies or antibody fragments that bind to IgE or to an IgE receptor), angiogenesis inhibitors, analgesic agents, and anti-infective agents.

In some embodiments the second agent is a neuroprotective agent or anti-oxidant or a compound that inhibits or slows down the visual cycle.

In certain embodiments of the invention the additional active agent is an angiogenesis inhibitor. A variety of angiogenesis inhibitors are of use. In certain embodiments of the invention the angiogenesis inhibitor specifically binds to one or more vascular endothelial growth factor (VEGF) isoforms or receptors. The angiogenesis inhibitor may be an antibody, antibody fragment, polypeptide, peptide, nucleic acid, aptamer, or siRNA. In certain embodiments of the invention the angiogenesis inhibitor specifically binds to one, more than one, or all vascular endothelial growth factor (VEGF) isoforms or receptors. In some embodiments the second therapeutic agent is a humanized mononclonal antibody that binds to vascular endothelial growth factor (VEGF), such as an antibody described in Presta, L G, et al., Cancer Res., 57, 4593-4599 (1997) or an antigen-binding fragment thereof or an antibody or antibody fragment one that binds to the same epitope. In certain embodiments of the invention the angiogenesis inhibitor is or comprises a mammalian peptide or polypeptide such as pigment epithelium-derived factor (PEDF), angiostatin, endostatin, etc., or a fragment thereof that retains anti-angiogenic activity. The angiogenesis inhibitor may be one that is recognized in the art as useful for treating AMD and/or CNV or RNV, such as Lucentis® (ranibizumab), Avastin® (bevacizumab), or Macugen® (pegaptanib sodium). In some embodiments, ranibizumab, bevacizumab, or pegaptanib is provided in a concentration or amount ranging from about 0.5 to about 5 times that approved for clinical use for wet AMD as a single agent. In exemplary embodiments, the amount is about 0.3 mg, 0.5 mg, 1.0 mg, or 1.5 mg. In some embodiments a liquid composition comprising a compstatin analog and an angiogenesis inhibitor is administered to an eye that exhibits choroidal neovascularization (CNV) and/or retinal neovascularization (RNV), e.g., an eye suffering from wet AMD. The inhibitor may be produced, e.g., using recombinant technology, hybridoma technology, chemical synthesis or isolated from naturally occurring sources.

The invention also provides a method comprising: (a) administering to a mammalian subject a compstatin analog in an amount sufficient to form a macroscopic, gel-like structure upon introduction into an extravascular location of the subject; and (b) administering an angiogenesis inhibitor to the subject. The angiogenesis inhibitor may or may not be administered in the liquid composition in different embodiments of the invention. The angiogenesis inhibitor, if admninistered in a different composition, may be administered to the same extravascular location or to a different location. If administered separately, the liquid composition may be administered prior to, at essentially the same time as, or following administration of the angiogenesis inhibitor. In some embodiments the angiogenesis inhibitor is administered first, and a liquid composition of the invention is administered after a time interval. The time interval may be, e.g., up to 1, 2, or 4 weeks after administration of the angiogenesis inhibitor, or up to 2 or 3 months after administration of the angiogenesis inhibitor. In some embodiments a liquid composition of the invention is administered to the eye of a subject with AMD after the subject experiences an improvement in visual acuity and/or exhibits reduced retinal thickness and/or reduced blood vessel leakage in the eye (e.g., as measured using optical coherence tomography or fluorescein angiography). The angiogenesis inhibitor can be used in standard doses and routes of administration for such agents (e.g., intravitreal administration).

The invention provides liquid compositions comprising: (a) a compstatin analog in an amount sufficient to form a macroscopic, gel-like structure upon introduction into an extravascular location of a mammalian subject; and (b) an additional complement inhibitor wherein said additional complement inhibitor is not a compstatin analog. In various embodiments the second complement inhibitor is a peptide, polypeptide, non-peptide small molecule, aptamer, antibody, or nucleic acid. In certain embodiments of the invention the second agent is a cyclic peptide. In certain embodiments the agent is an antagonist of a C5a receptor (C5aR). Exemplary C5a receptor antagonists include a variety of small cyclic peptides such as those described in U.S. Pat. No. 6,821,950; U.S. Ser. No. 11/375,587; and/or PCT/US06/08960 (WO2006/099330). In certain embodiments of the invention a cyclic peptide comprising the sequence [OPdChaWR] (SEQ ID NO: 33) is used. In certain embodiments of the invention a cyclic peptide comprising the sequence [KPdChaWR] (SEQ ID NO: 37) is used. In certain embodiments a peptide comprising the sequence (Xaa)ₙ[OPdChaWR] (SEQ ID NO: 38) is used, wherein Xaa is an amino acid residue and n is between 1 and 5. In certain embodiments a peptide comprising the sequence (Xaa)ₙ[KPdChaWR] (SEQ ID NO: 39) is used, wherein Xaa is an amino acid residue and n is between 1 and 5. In certain embodiments of the invention n is 1. In certain embodiments of the invention n is 1 and Xaa is a standard or nonstandard aromatic amino acid. For example, the peptides F-[OPdChaWR] (SEQ ID NO: 40), F-[KPdChaWR] (SEQ ID NO: 41), Cin-[OPdChaWR] (SEQ ID NO: 42), and HCin-[OPdChaWR] (SEQ ID NO: 43) are of interest. Optionally the free terminus comprises a blocking moiety, e.g., the terminal amino acid is acetylated. (Abbreviations: O: ornithine; Cha: cyclohexylalanine; Cin: cinnamoyl; Hcin: hydrocinnamoyl; square brackets denote internal peptide bond).

Particle-Containing Compositions

In certain embodiments the liquid composition comprises, in addition to the compstatin analog in sufficient amounts to form a gel-like structure, a population of particles that comprise a therapeutic agent, wherein the composition is capable of releasing the therapeutic agent. Such compositions, and gels comprising such compositions, are an aspect of the invention. The particles may be, e.g., microparticles or nanoparticles. The microparticles or nanoparticles may be polymer-based particles (e.g., containing synthetic organic polymers, polypeptides, etc.), lipid-based or lipid-containing particles such as liposomes, niosomes, micelles, etc. In certain embodiments of the invention the therapeutic agent is a complement inhibitor. The particles are retained in the gel-like structure and released over time as it disintegrates or dissolves. The particles may at least in part disintegrate or dissolve while entrapped in the gel. The complement inhibitor may be a compstatin analog, which may be either the same compstatin analog as that forming the gel-like structure or a different compstatin analog. Other useful therapeutic agents are discussed above.

Nanoparticles or microparticles can be made using any method known in the art including, but not limited to, spray drying, phase separation, single and double emulsion, solvent evaporation, solvent extraction, and simple and complex coacervation. Particulate polymeric compositions can also be made using granulation, extrusion, and/or spheronization. See, e.g., U.S. Publication No. 20040092470. Methods for making liposomes and other lipid-based particles are known in the art. In some embodiments the particles consist essentially of one or more compstatin analogs. In some embodiments the particles are composed of at least 50% compstatin analog(s) by dry weight. Optionally, such particles contain one or more excipients.

A composition can contain nanoparticles or microparticles having different compositions and/or properties. The conditions used in preparing the particles may be altered to yield particles of a desired size or property (e.g., hydrophobicity, hydrophilicity, external morphology, density, hardness, “stickiness”, shape, etc.). The method of preparing the particle and the conditions (e.g., solvent, temperature, concentration, air flow rate, etc.) used may also depend on the therapeutic agent and/or the composition of the polymer matrix. It is generally desirable to avoid extremes of temperature or pH that could result in significant degradation of the complement inhibitor. It will be appreciated that the extent of degradation may be a function of both the particular conditions and the time over which the complement inhibitor is exposed to the conditions, as well as the structure and properties of the agent itself For example, a stable peptide such as a compstatin analog may have significant advantages. Compositions can be tested to determine whether the method selected is appropriate in terms of retaining sufficient efficacy. In certain embodiments a selected formulation method results in a composition in which, following formulation, the compound retains at least 10% preferably at least 20%, 50%, or more of the level of activity of the input compound.

The method of preparing the particle and the conditions (e.g., solvent, temperature, concentration, air flow rate, etc.) used may also depend on the particular active agents and other components included in the composition. If the particles prepared by any of the above methods have a size range outside of the desired range, the particles can be sized, for example, using a sieve, by milling, etc. Combinations of methods may be employed.

Microparticles and nanoparticles of use in the invention can have a range of dimensions. Generally, a microparticle will have a diameter of 500 microns or less, e.g., between 1 and 500 microns, between 50 and 500 microns, between 100 and 250 microns, between 20 and 50 microns, between 1 and 20 microns, between 1 and 10 microns, etc., and a nanoparticle will have a diameter of less than 1 micron, e.g., between 10 nm and 100 nm, between 100 nm and 250 nm, between 100 nm and 500 nm, between 250 nm and 500 nm, between 250 nm and 750 nm, between 500 nm and 750 micron. 30. In some embodiments the microparticles have a diameter ranging from 5-750 microns. In some embodiments the microparticles have a diameter ranging from 10 to 500 microns. In some embodiments the microparticles have a diameter ranging from 20 to 200 microns. In some embodiments the nanoparticles have a diameter ranging from 5-750 nanometers. In some embodiments the nanoparticles have a diameter ranging from 10 to 500 nanometers. In some embodiments the nanoparticles have a diameter ranging from 20 to 200 nanometers. In some embodiments the size is selected to minimize or avoid transport across capillary walls, thereby minimizing entry into the vascular system.

In some embodiments the microparticles or nanoparticles are formed from a polymer selected from the group consisting of hyaluronan, chitosan, collagen, gelatin, alginate, poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, blends and copolymers thereof. For example, the polymer could be polylactic acid (PLLA), polyglycolic acid (PGA) or PLGA. Particles can be substantially uniform in size (e.g., diameter) or shape or may be heterogeneous in size and/or shape. They may be substantially spherical or may have other shapes, in which case the relevant dimension will be the longest straight dimension between two points on the surface of the particle rather than the diameter. The particle population can consist of between about 20% and about 100% particles falling within any of the afore-mentioned size ranges, e.g. about 40%, 40%, 50%, 60%, 70%, 80%, 90%, etc.

Liquid Compositions and Methods of Producing Them

In general, complement inhibitors and other therapeutic agents are manufactured using standard methods known in the art and suitable for compounds of that class. Peptides such as compstatin analogs and other peptides discussed herein may be manufactured using standard solid phase peptide synthesis techniques. For example, a compstatin analog can be produced by solid phase synthesis of the protected peptide using Fmoc chemistry, leavage of the peptide from the resin, together with the removal of the side chain protecting groups, disulfide bond formation between Cys2 and Cys12, followed by purification, and conversion of the oxidized peptide to the acetate form. If desired, the bulk product is lyophilized. Peptide manufacturers include companies such as Advanced Chemtech, Ambiopharm, American Peptide, Dalton Pharma Services, GenScript, Integrated Biomolecule, Lonza, New England Peptide, Peptide 2.0, Synthetech, etc. Recombinant polypeptides may be produced using standard recombinant nucleic acid techniques as described, e.g., in U.S. Ser. No. 11/247,886 and PCT/US2005/36547 (WO2006042252). See, e.g., Hardin, C., et al., (Eds.), “Cloning, Gene Expression and Protein Purification: Experimental Procedures and Process Rationale”, Oxford University Press, Oxford, 2001, for further information regarding production of recombinant polypeptides and purification of polypeptides. Antibodies, e.g., monoclonal antibodies, may be harvested from hybridomas or produced using recombinant methods as known in the art. Nucleic acids, e.g., siRNAs, aptamers, etc., are synthesized using standard methods. Chemical modifications such as pegylation may be performed using standard methods.

A liquid composition of the present invention comprises a compstatin analog and a pharmaceutically acceptable carrier. The term “pharmaceutically acceptable carrier” refers to a non-toxic carrier that does not destroy the pharmacological activity of the compound with which it is formulated. Pharmaceutically acceptable carriers or vehicles that may be used in the compositions of this invention include, but are not limited to, liquids such as water, physiological saline, and the like. In certain embodiments of the invention the pharmaceutically acceptable carrier is water. In some embodiments the pH of the liquid composition is between 3.5 and 6.5. In some embodiments the pH of the liquid composition is between 4.0 and 4.5. In some embodiments the pH of the liquid composition is between 4.5 and 5.0. In some embodiments the pH of the liquid composition is between 5.0 and 5.5. In some embodiments the pH of the liquid composition is between 5.0 and 6.5.

The liquid composition can contain a variety of additional constituents in certain embodiments of the invention. For example, buffers, pH modifiers, solubilizing agents, osmolarity-adjusting agents (e.g., sugars), etc., can be included. Standard excipients known in the art can be employed. In some embodiments, a compstatin analog is dissolved in a liquid medium, e.g., water, to which one or more buffers or excipients has been added. For example, a solution containing an excipient such as an amino acid or polyol (e.g., a sugar alcohol) and/or a buffer is prepared. Compstatin analog is added in powder form and dissolved. The solution may be filtered if desired.

Pharmaceutically acceptable salts of the compstatin analog can be used, such as those derived from pharmaceutically acceptable inorganic and organic acids and bases. Furthermore it will be appreciated that the invention encompasses solvates, hydrates, enantiomeric forms, conformers, tautomers, polymorphic forms, etc., of the active agents described herein. In some embodiments the compstatin analog is provided with acetate as a counterion.

The amount and concentration of the compstatin analog(s) in a composition can vary depending on a number of factors including, but not limited to, the identity of the compstatin analog(s), the condition being treated and its severity, etc., provided that the amount and concentration are sufficient to result in formation of a macroscopic, gel-like structure when the composition is administered. The duration of release can be regulated by appropriate selection of the amount and/or concentration of compstatin analog administered. It will also be appreciated that the minimum amount and/or concentration of the compstatin analog required to form a gel-like deposit may vary depending on factors such as the species, age of the subject, etc. One of skill in the art can readily determine the appropriate values. Furthermore, it is not necessary that the composition form a gel-like deposit in every individual treated.

In some embodiments, the invention provides a composition comprising a compstatin analog, wherein the composition is characterized in that, upon intravitreal administration to a primate the composition forms a gel that remains detectable by ultrasound and/or ophthalmological examination for at least 3 months in at least 75% (e.g., at least 80%, at least 85%, at least 90%, at least 95%, or more) of eyes to which the composition is administered. In some embodiments, the invention provides a composition comprising a compstatin analog, wherein the composition is characterized in that, upon intravitreal administration to a primate the composition forms a gel that remains detectable by ultrasound and/or ophthalmological examination for at least 6 months in at least 75% of eyes to which the composition is administered. In some embodiments, the invention provides a composition comprising a compstatin analog, wherein the composition is characterized in that, upon intravitreal administration to a primate the composition forms a gel that remains detectable by ultrasound and/or ophthalmological examination for at least 9 months in at least 75% of eyes to which the composition is administered. In some embodiments the primate is a non-human primate, e.g., a cynomolgus monkey. In some embodiments the primate is a human. In some embodiments of the invention, the gel becomes undetectable by ultrasound within 12, 15, 18, or 24 months of administration in at least 75% (e.g., at least 80%, at least 85%, at least 90%, at least 95%, or more) of eyes. In some embodiments, the gel releases between 1μg and and 5μg compstatin analog daily, e.g., about 3 μg daily, over at least 3 months, e.g., over 3-6, 6-9, or 9-12 months.

The invention provides liquid compositions comprising a compstatin analog and one or more excipients or buffers. In some embodiments, the excipient is an amino acid. In some embodiments the amino acid is arginine, histidine or serine. In some embodiments the excipient is a “sugar alcohol” (also referred to as a “polyhydric alcohol”). In some embodiments the excipient is selected from the group consisting of: glycol, glycerol, erythritol, arabitol, xylitol, ribitol, mannitol, sorbitol, isomalt, maltitol, and lactitol. In some embodiments, the excipient is present at a concentration ranging from 1 mM to 250 mM, e.g., between 10 mM and 100 mM or between 10 mM and 50 mM, e.g., about 20-45 mM. In some embodiments the composition comprises two or more excipients. In some embodiments the buffer is selected from an acetate buffer and a phosphate buffer. In some embodiments the buffer is sodium acetate. In some embodiments the buffer concentration is between 10 mM and 1 M, e.g., between 20 mM and 500 mM, e.g., between 50 mM and 200 mM.

As described in Example 4, it was observed that presence of certain excipients and/or buffers in the liquid composition resulted in gels that have a different and more fragile consistency than gels formed from compositions containing the same amount of the compstatin analog and water only. Furthermore, such gels disappeared more rapidly following intravitreal administration than did gels formed from compositions containing the same amount of the compstatin analog and water only. The inventors recognized that such excipients and buffers provide a valuable means of modulating various physical properties of the gel and modulating the rate at which compstatin analog is released from the gel in vivo. The invention provides a method of modulating the rate of release of a compstatin analog from a gel containing said compstatin analog, the method comprising providing said compstatin analog in a liquid composition comprising an excipient or buffer, wherein presence of the excipient or buffer modulates (e.g., increases or decreases) the rate at which a gel formed upon in vivo administration of the composition dissolves or degrades.

One aspect of the invention is the recognition that excipients that increase the rate of dissolution/disintegration of the gel in vivo can permit increased amounts of compstatin analog to be administered and/or administration of increased concentrations of compstatin analog, while avoiding formation of a gel that is too stable to release desired amounts of compstatin analog over time.

Measuring Complement Inhibition

Any suitable method can be used for assessing the ability of an agent to inhibit complement activation (or any other relevant properties). A number of in vitro assays can be used. For example, ability of an agent to inhibit the classical or alternative complement pathway may be assessed by measuring complement-mediated hemolysis of erythrocytes (e.g., antibody-sensitized or unsensitized rabbit or sheep erythrocytes), by human serum or a set of complement components in the presence or absence of the agent. An agent inhibits complement if it decreases hemolysis in this inhibition assay to a statistically significant degree (p<0.05). The ability of an agent to bind to one or more complement component such as C3, C5, factor B, factor D can be assessed using isothermal titration calorimetry or other methods suitable for performing in liquid phase. In another embodiment, the ability of an agent to bind to a complement component is measured using an ELISA assay. For example, the wells of a microtiter plate are coated with the agent. A complement inhibitor can be functionalized in order to facilitate binding it to a plate. For example, the agent could be biotinylated, and a streptavidin-coated plate is used. Complement component(s) are added to the wells. After a period of incubation the wells are washed, and bound complement components are detected using antibodies to the complement component of interest. Other methods of use include surface plasmon resonance, equilibrium dialysis, etc.

Methods for measuring systemic or local complement activation taking place in vitro or in vivo and for determining the ability of a complement inhibitor to inhibit such activation are known in the art. For example, measurement of complement activation products such as C3a, C5a, C3bBb, C5b-9, covalent complexes between the recognition molecule of the classical pathway (C1q) and activated C4, etc., provides an indication of the extent of complement activation. A decrease in the amount of such products indicates inhibition of complement activation in certain embodiments of the invention. In some embodiments a ratio between an active cleavage product and its inactive desArg form is measured (e.g., C3a/C3adesArg). One of skill in the art can distinguish between classical, alternative, and lectin pathway activation by appropriate selection of the complement activation product(s) measured and/or appropriate activators of complement such as zymosan, lipopolysaccharide, immune complexes, etc. Other methods involve measuring complement-mediated hemolysis of red blood cells as a result of terminal complex formation.

Complement activation in vivo and/or its inhibition by a complement inhibitor, can be measured in an appropriate biological sample. For example, systemic complement activation and/or its inhibition by a complement inhibitor, can be measured in a blood or plasma sample. Local activation and/or inhibition in the vitreous humor can be measured in a sample of vitreous humor. Local activation and/or inhibition in the respiratory tract can be measured in a sputum sample. Local activation and/or inhibition in a joint can be measured in a sample of synovial fluid. Local activation and/or inhibition in the CNS can be measured in a sample of CSF. Serial measurements beginning before administration of a complement inhibitor provide an indication of the extent to which the complement inhibitor inhibits complement activation and the time course and duration of the inhibition. It will be appreciated that a decrease in activation products may only become apparent once activation products present prior to administration of the complement inhibitor have been degraded or cleared.

Suitable methods are described in a number of references cited herein (U.S. Pat. Nos. 5,157,110; 6,551,595; U.S. Pat. No. 6,319,897; WO2004/026328 (PCT/US2003/029653), U.S. Ser. No. 10/937,912; Morikis, 2004; Mallik, 2005; Katragadda, M., 2006.

Monitoring Degradation of a Gel-like Deposit Comprising a Compstatin Analog

As noted above, certain gel-like deposits of the present invention are detectable in vivo by ultrasound. If desired, degradation of the deposit could also be assessed by including a detectable moiety within the liquid composition to be administered. As used herein a “detectable moiety” is a moiety, e.g., molecule or supramolecular complex, which can be detected when present in vivo by a particular method or methods of interest. Typically the detection method is external and non-invasive, i.e., the method does not involve penetration of the skin or another externally accessible body surface or entry into the body. In certain embodiments, detection of the detectable moiety can be used to assess the mass or volume of the sustained release formulation or device that remains intact at a time “X” after administration and/or to assess the mass or volume of the sustained release formulation or device that has degraded at a time “X” after administration. If it is determined that a predetermined mass or volume of the formulation has degraded or remains, the subject may be retreated within a suitable time period. For example, retreatment can be scheduled to take place within 1, 2, 3, or 4 weeks of the time when the formulation is determined or expected to be at least 70%, 80%, 90%, 95%, 99% or 100% degraded. Detection of the detectable moiety can alternately or additionally be used to assess the amount of therapeutic agent that remains within the sustained release formulation or device, i.e., has not yet been released.

Methods of Treatment and Patient Selection

The invention provides methods of treating a subject comprising administering a liquid composition comprising a compstatin analog to an extravascular location of the subject in an amount sufficient to form a macroscopic, gel-like deposit. The methods of the invention may include providing a subject to which a composition of the invention is to be administered. The subject is typically at risk of or suffering from a disorder, e.g., a complement-mediated disorder. In certain embodiments the subject is at risk of or suffers from macular degeneration, e.g., age-related macular degeneration. In some embodiments the subject suffers from at at least one complement-mediated disorder characterized by ocular inflammatrion. In certain embodiments the subject is at risk of or suffers from at least one complement-mediated disorder in addition to an eye disorder characterized by macular degeneration or ocular inflammation.

The composition is typically administered to the subject with the intent of treating or preventing development of such disorder. Thus the subject will typically have been identified as being at risk of or suffering from such a condition. Any suitable tests and criteria can be used to identify a subject at risk of or suffering from disorder of interest herein. Methods for diagnosis of the disorders of interest herein and for assessing response to therapy are known in the art.

In certain embodiments of the invention the method of treatment comprises determining whether the subject has a genetic polymorphism that is associated with increased risk of developing or having the disorder. “Determining” as used here refers to establishing that a subject has a polymorphism that increases the risk of the dirsorder, either by performing or ordering a suitable test, or by receiving results of a test performed or ordered by another, wherein the test ascertains whether the subject has the polymorphism. It will be appreciated that a useful genetic test need not be 100% accurate. The polymorphism may be in a gene that encodes a complement component.

Genetic studies have demonstrated association between certain alleles of genes encoding various complement-related proteins and increased susceptibility to developing AMD and/or increased likelihood of developing a severe form of AMD. An allele that is associated with increased likelihood of developing a disorder or condition and/or increased likelihood of developing a severe form of the disorder or condition or having a poor outcome from the disorder or condition is referred to herein as a “risk allele” for that disorder or condition, and the gene is referred to as a “risk modifier” for the disorder or condition. Certain risk alleles are alleles of the gene that encodes complement factor H (CFH), wherein the alleles contain a polymorphism resulting in a CFH isoform that contains His rather than Tyr at position 402 (Tyr402His polymorphism). Without wishing to be bound by any theory, the Tyr402His variant of CFH may be less effective at controlling complement activation and/or may have altered tissue localization adversely affecting its complement control ability. Subsequent studies have found that other CFH isoforms are tightly associated with AMD risk (Klein, R. J. et al. Complement Factor H Polymorphism in Age-Related Macular Degeneration. Science (2005); Edwards, A. O. et al. Complement Factor H Polymorphism and Age-Related Macular Degeneration. Science (2005); Haines, J. L. et al. Complement Factor H Variant Increases the Risk of Age-Related Macular Degeneration. Science (2005). Furthermore, variants of the genes that encode complement proteins C2, C3, factor B, C7 and MBL-2 have also been associated with AMD risk (Gold, B. et al. Variation in factor B (BF) and complement component 2 (C2) genes is associated with age-related macular degeneration. Nat. Genet. 38, 458-462 (2006); Dinu, V. et al. Evidence for Association between Multiple Complement Pathway Genes and AMD. Genet. Epidem. 31, 224-237 (2007) Yates, J. R. W., Complement C3 Variant and the Risk of Age-Related Macular Degeneration, N. Engl. J. Med., 357: 19-27, 2007). In addition, variants of genes that encode CFHR1, CFHR3, and CFI have been associated with AMD risk and methods based at least in part on detection of such variants may be of use.

The present invention encompasses assessing the genotype of a subject with respect to any of the genes and/or polymorphisms described in the afore-mentioned references, or any other gene(s) encoding a complement-related protein (see above) and selecting the subject as a suitable subject for administration of a liquid composition comprising a compstatin analog based at least in part on the results of said assessment (e.g., if the assessment indicates that the subject is at increased risk of developing or having a complement-mediated disorder, e.g., AMD, the composition is administered). It will be appreciated that polymorphisms, e.g., SNPs, may be in linkage disequilibrium (LD) with other polymorphisms, e.g., other SNPs, located on the same chromosome. Such SNPs may be present in haplotypes. For example, some SNPs may be linked over distances of up to 100 kB or even over 150 kB or more (Reich, D. E., et al., Nature, 411, 199-204, 2001). Thus in some embodiments the methods of the invention comprise determining whether an individual has a haplotype that comprises at least one polymorphic variant associated with increased risk of a complement mediated disorder, wherein said polymorphism is in a complement-related gene. In some embodiments the haplotype comprises the Tyr402His coding variant of the CFH gene. In some embodiments the haplotype is a CFH haplotype that does not comprise the Tyr402His coding variant (see, e.g., Li, et al., supra).

The genotype of the individual can be determined using any of a variety of methods. The particular method employed is not critical to the present invention and need not be described here in detail, such methods being well known in the art. The methods typically utilize a biological sample obtained from the individual, wherein the sample comprises nucleic acids and/or proteins. As used herein, a “biological sample” refers to any of the following: a cell or cells, a portion of tissue, a body fluid such as blood, urine, saliva, cerebrospinal fluid, etc. The term “biological sample” also includes any material derived by processing a biological sample as previously defined, e.g., by isolating or purifying DNA, RNA, and/or protein, from the sample, by subjecting the sample or a portion thereof to amplification, restriction enzyme digestion, etc. Typically a blood or tissue sample is used. Methods can involve testing the individual's DNA to determine whether the DNA comprises an allele or polymorphism of interest. RNA can also be used, if the polymorphism of interest lies in a portion of the gene that is transcribed. In some embodiments the methods involve determining the identity of a particular nucleotide, wherein polymorphism(s) at the position of such nucleotide are associated with increased risk of poor outcome following trauma and/or increased susceptibility to AMD or another complement-mediated disorder.

Methods for performing such tests are well known in the art and include, e.g., isolating the DNA or RNA, optionally amplifying it (e.g., using the polymerase chain reaction (PCR) or reverse transcriptase-PCR), and performing a variety of methods such as allele-specific primer extension, allele-specific hybridization, restriction enzyme digestion, sequencing, etc. In some embodiments genotyping is performed using a microarray, or “chip”. In some embodiments genotyping is performed using a bead-based assay such as the Luminex platform. Other methods of use include oligonucleotide ligation assays (U.S. Pat. Nos. 5,185,243, 5,679,524 and 5,573,907), cleavage assays, heteroduplex tracking analysis (HTA) assays, etc. Examples include the Taqman® assay, Applied Biosystems (U.S. Pat. No. 5,723,591). Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader® assays (Third Wave Technologies), described in Eis, P. S. et al., Nat. Biotechnol. 19:673, 2001, can also be used. Assays based on molecular beacons (U.S. Pat. Nos. 6,277,607; 6,150,097; 6,037,130) or fluorescence energy transfer (FRET) may be used. U.S. Pub. No. 20050069908 and references therein describe a variety of other methods that can be used for the detection of nucleic acids. U.S. Pat. Nos. 5,854,033, 6,143,495, and 6,239,150 describe compositions and a method for amplification of and multiplex detection of molecules of interest involving rolling circle replication. The method is useful for simultaneously detecting multiple specific nucleic acids in a sample. Optionally the nucleic acids are sequenced. U.S. Pub. No. 20050026180 describes methods for multiplexing nucleic acid reactions, including amplification, detection and genotyping, which can be adapted for determining the sequence at specific locations of interest for purposes of determining whether an individual has a genotype associated with increased risk of poor outcome following trauma.

In summary, and without limitation, suitable methods include hybridization-based methods such as dynamic allele-specific hybridization, use of molecular beacons, SNP microarrays, enzyme-based methods such as those based on restriction fragment length polymorphism, PCR-based methods, methods employing flap endonuclease, primer extension, 5′-nuclease, oligonucleotide ligase assay, other post-amplification methods based on physical properties of DNA, single strand conformation polymorphism, temperature gradient gel electrophoresis, denaturing high performance liquid chromatography, and sequencing. High throughput sequencing is becoming more efficient at a rapid pace, and it is envisioned that sequencing may become routinely used for genotyping purposes. Methods based on pyrosequencing, in situ sequencing, bead-based sequencing (US2007087362), etc., are of use. See also PCT/US2006/029449 (WO2007014338) for further information and related definitions.

In certain embodiments the solution is administered directly to the eye, e.g., by intraocular injection. Standard methods of intraocular administration can be used such as intravitreal injection, injection into the anterior chamber, etc. In certain embodiments of the invention the composition is administered by sub-tenon injection, retrobulbar injection, or subconjunctivally.

In some embodiments, a subject suffering from ocular neovascularization (e.g., a subject with wet AMD or proliferative diabetic retinopathy) is treated with an angiogenesis inhibitor in order to inhibit bleeding and/or fluid leakage prior to administering a composition of the invention. In some embodiments, a subject is treated with an angiogenesis inhibitor, and a composition of the invention is administered between 1 and 6 weeks following administration of the angiogenesis inhibitor.

In certain embodiments the solution is administered in or near a joint.

Delivery can be accomplished by injection (e.g., using a 25, 27, or 30 gauge needle or the like), by catheter, etc.

In certain embodiments of the invention the composition containing a compstatin analog is delivered to one or more of the CSF-containing cavities or chambers of the central nervous system, e.g., the ventricles or cisterna magna. To deliver an agent to a ventricle or the cistema magna using an infusion pump, the catheter may be implanted so that the discharge portion lies in the ventricle or the cisterna, resulting in formation of a gel-like deposit therein. The compstatin analog agent diffuses out of the ventricle or cistema magna. Delivery to these locations therefore allows delivery of the agent to a relatively wide area of the brain rather than localizing it more closely to a specific site. In certain embodiments of the invention delivery to a CSF-containing space is accomplished by surgically implanting a catheter through the skull so that the tip has access to the space. The other end of the catheter is then connected to a reservoir (e.g., an Ommaya reservoir), which is placed beneath the scalp (subcutaneously).

Methods for intrathecal administration are well known in the art. If the subject suffers from spinal cord injury, the catheter is implanted so that the discharge portion lies in the intrathecal space while the other end is connected to the pump reservoir. Such methods are commonly used in the treatment of chronic pain, and are routinely used to deliver analgesic agents over a period of months. Similar methods are of use in the present invention to deliver a liquid composition comprising a compstatin analog in an amount effective to form a macroscopic, gel-like deposit in the intrathecal space.

In one embodiment of the present invention a subject suffering from traumatic brain injury, stroke, or spinal cord injury, is treated by administration of a compstatin analog under conditions appropriate for formation of a macroscopic, gel-like deposit comprising the compstatin analog. In certain embodiments a neuroprotective or neurotropic agent is also administered systemically and/or locally in various embodiments of the invention. In certain embodiments the neuroprotective or neurotropic agent is administered together with the complement inhibitor in a single composition.

The dosing interval (i.e., the time between individual administrations of an inventive composition) and the dose of the compstatin analog delivered with each administration can vary. In certain embodiments the composition is delivered at times more than 6 weeks apart, e.g., 2, 3, 4, 5, or 6 months apart, or any intervening number of weeks, e.g., 8, 10, 12, 14, 16 weeks, etc. In other embodiments the composition is delivered at even greater time intervals, e.g., at times 7, 8, 9, 10, 11, or 12 months apart. In other embodiments the time interval is 6 weeks or less, e.g., 1, 2, 3, 4, 5, or 6 weeks apart. For example, the composition may be administered on average every 2 weeks, every 4 weeks, every 30 days, etc. Of course the time interval can vary. For example, the time intervals between doses can alternate between 6 weeks or less and more than 6 weeks. In certain embodiments the average time interval between administrations of an inventive composition is at least 6 weeks, e.g., 2, 3, 4, 5, or 6 months, or any intervening number of weeks, e.g., 8, 10, 12, 14, 16 weeks, etc. In certain embodiments of the invention the composition is administered multiple times at time intervals on average at least 6, 8, 10, or 12 weeks apart, or on average 3, 4, 6, 8, 12, 15, 18, or 24 months apart, etc. The composition may be administered at least 1, 2, 5, 10, 15, 20, or more times. The composition may be administered indefinitely at various intervals to a subject suffering from or at risk of a complement-mediated disorder, e.g., AMD.

### EXAMPLES

### Example 1

### Formation of a Gel-Like Deposit Upon Intravitreal Administration of a Potent Compstatin Analog to Non-Human Primates

Compstatin Analog Synthesis

The synthesis of a potent compstatin analog (also referred to in Examples 1-3 as “compound”) was accomplished following the solid phase methodology described by Merrifield (J. Amer. Chem. Soc. 85, 2149 (1963)). The α-amino group of each amino acid was protected with Fmoc groups. Side chain functional groups were also blocked with various appropriate protective groups. The peptide chain was formed by derivatization of the c-terminal amino acid (i.e. Thr) onto the Rink Amide AM resin, followed by sequential coupling of amino acids, removal of side chain protecting groups, and cleavage from the resin. When the full peptide sequence was completed, the N-terminus of the peptide resin was acetylated (using a capping solution comprised of Ac₂O/CH₂Cl₂/DIEA in a 6:50:3 v/v ratio), then the resin was rinsed with successive volumes of MeOH and DMF. Following cleavage from the resin using standard methods, the disulfide bridge between the Cys² and Cys¹² residues of the peptide was formed by I₂ oxidation. First, the linear peptide was dissolved in 20% ACN in water to make a peptide solution with a concentration of 1 mg/mL. Second, the I₂/NaI solution was added dropwise to the peptide solution while stirring (NaI is used to increase the solubility of I₂ in H₂O). At the beginning of the addition, the yellow color of I₂ disappeared upon contact. Once the yellow color of I₂ remained, a sign that enough I₂ had been added, the flask was stirred another 30 minutes to complete the oxidation. An ascorbic acid solution (0.1 M) was added to neutralize the excess of I₂ and quench the reaction. The reaction was monitored with the in-process control high pressure liquid chromatography (HPLC) method.

After the oxidation was completed, the reaction mixture was then prepared for preparative HPLC purification by filtering through a 1 μm glass fiber filter. The filtered peptide solution was loaded onto a preparative HPLC column packed with C₁₈ reverse phase resin which was operated by a preparative HPLC system (Varian HPLC). The column was eluted with Buffer “A” [0.1% TFA in H₂O—1:1000 (v/v)] and a linear gradient using Buffer “B” [0.1% TFA in acetonitrile—1:1000 (v/v)].

The fractions that were collected from the preparative column were analyzed by an analytical HPLC system (Varian HPLC) equipped with an analytical HPLC column (Kromasil C18 5 μm). Fractions that met purity requirements were then pooled for the next process step. Fractions that did not meet the purity requirement were re-purified to reach the purity requirement, and then pooled. The process was designed to achieve at least 95% purity. The pooled purified fractions were then reloaded on the preparative HPLC column, washed with ammonium acetate buffer, and eluted with the desired buffer system to exchange the peptide salt form into the desired salt, in this case, as the acetate salt. Again fractions were analyzed by analytical HPLC and those fractions that met the final purity criteria were pooled and prepared for lyophilization. A manifold-type lyophilizer was used, and no more than 350 mL of the purified peptide solution was placed in each lyophilizer jar after being frozen. The freeze-drying took place over at least 3 days. The final bulk peptide was evaluated for purity using reversed phase HPLC.

Manufacture of Liquid Compositions

Two different formulations were manufactured by dissolving the compound into water for injection at different concentrations: (i) 0.299 mg/mL, designated LD and (ii) 4.51 mg/mL, designated as HD and filter sterilizing through a Millipore Durapore 47 mm 0.22 μm filter. The liquid compositions were dispensed into individual vials (250 μl each). The pH of the LD composition was 5.48 while that of the HD composition was 6.0.

Intravitreal Administration

During studies involving intravitreal administration of the compstatin analog whose synthesis was described above (SEQ ID NO: 32) in water for injection to non-human primates it was observed that the compound has the ability to form gel-like, approximately spheroidal intravitreal deposits in the primate (Cynomolgus monkey) eye following intravitreal injection. The deposits formed at the injection site and only with the higher of the two concentrations tested. The high dose consisted of the intravitreal (IVT) injection of an estimated dose of 150 μg of compound in a volume of 50 μL water for injection while the low dose consisted of an estimated dose of 3 μg of compound in a volume of 50 μL water for injection. Slit lamp examinations of the anterior segment of the eye showed no abnormalities on days 2 and 15 following administration. Binocular indirect ophthalmoscopy on day 2 demonstrated diffuse vitreal haze in the right eye of one animal dosed IVT at 3 μg/eye, and in four animals dosed IVT at 150 μg/eye. Vitreal haze was still present on day 15 in four of these animals and another two animals were noted exhibiting vitreal haze for the first time. There was no evidence that the compound within the vitreous cavity caused any apparent adverse reactions. ERG values were within normal limits and the tonometry indicated no drug-related changes in intraocular pressure attributed to compound administration.

At necropsy, in the majority of male animals (10 of 12) that had been dosed with compound IVT at 150 μg/eye, gel-like spheroidal intravitreal deposits were observed upon dissection of the eye. These deposits were isolated and analyzed for the presence of the compound. Results indicated that compound content ranged from 7.0 μg up to 72 μg per deposit. One of two animals sacrificed at 2 weeks post single dose IVT injection showed no spheroidal intravitreal deposit and thus no detectable compound was observed. Similarly, one animal dosed IVT at 150 μg/eye that was sacrificed 4 hours post treatment did not exhibit a spheroidal intravitreal deposit. Thus, the deposits were first observed at the first time point, four hours post treatment, and were still present in one of two animals sacrificed at the end of two weeks. Histopathologically, no drug-related abnormalities were observed in the eyes of any animal dosed with the compound, with or without the presence of intravitreal deposits.

The deposits were analyzed for drug content, and drug content was observed and measured using HPLC. The deposits were found to contain substantial amounts of compound. The vitreous of all animals dosed intravitreally with the compound at the 150 μg/eye dose level revealed the presence of the compound at all time points examined (from 4 hours to 2 weeks post administration). These results suggest that the deposits, when formed, released the compound over time.

### Example 2

### Characterization of Gel-Like Deposit Formed Upon Intravitreal Administration of a Potent Compstatin Analog to Rabbits

A study using New Zealand White (NZW) rabbits was performed to characterize deposit formation in more detail, to find the minimum compound concentration at which deposit formation occurred after intravitreal injection, and to assess the acute toxicological properties of the compound following intravitreal injection.

Methods

Compound Solution Preparation

HD composition was produced as described in Example 1 and diluted with WFI under sterile conditions to reach different doses of compound. A 50 μl volume was injected intravitreally into the rabbit eye.

Animals Treated

The study examined the ability of compound in amounts ranging from 0 to 200 μg/eye (0, 25, 50, 75, 100, 125, 150,175, 200 μg) to form deposits in the eye following intravitreal injection (Table 2). Each concentration was injected into three eyes. Thirteen animals were used for dose-ranging. Five additional animals were used for histological analysis.

Animal procedures

Anesthesia: Animals were anesthetized by intramuscular injection of a Ketamine (50 mg/kg)+xylazine (10 mg/kg) solution.

TABLE 2
Dose-ranging Study for Vitreous Deposits Formation in
New Zealand White Rabbits

Compound IVT
(μg/eye)

| Left eye | Right eye | Procedure^{A} | Animal ID |
|---|---|---|---|
| 25 | 25 | OE, DIS | 1312 |
| 25 | 50 | OE, DIS | 1334 |
| 50 | 50 | OE, DIS | 1335 |
| 75 | 75 | OE, DIS | 1344 |
| 75 | 100 | OE, DIS | 1345 |
| 100 | 100 | OE, DIS | 1346 |
| 125 | 125 | OE, DIS | 1313 |
| 125 | 150 | OE, DIS | 1310A |
| 150 | 150 | OE, DIS | 1338 |
| 175 | 175 | OE, DIS | 1339 |
| 175 | 200 | OE, DIS | 1340 |
| 200 | 200 | OE, DIS | 1341 |
| 0 | 0 | OE, DIS | 1342 |
| 0 | 0 | OE, HISTO | 1390 |
| 0 | 0 | OE, HISTO | 1391 |
| 25 | 25 | OE, HISTO | 1337 |
| 50 | 50 | OE, HISTO | 1343 |
| 100 | 100 | OE, HISTO | 1336 |

^{A}These procedures were done 27 hours after treatment.
OE = Ophthalmic Exams
DIS = Dissection
HISTO = Histopathology analysis

Pre-injection procedures: Eyes were prepared by instilling 2 drops of topical anesthetic (0.5% procainamide) followed by 2 drops of 5% povidone iodine ophthalmic solution directly on the eye. Special care was taken to cover the planned injection site with the povidone iodine solution. Excess liquid from the periocular area was blotted dry with a 4×4 gauze pad. Local anesthetic and povidone solution were allowed to act for at least 5 minutes before the intravitreal injection was performed.

Injection Procedures: Intravitreal injections were administered using a 0.5 cc tuberculin syringe (with permanently attached 29 G ½ inch needle); 0.2 ml of compound solution was withdrawn from the vial. Excess liquid was expelled, so that the syringe contained only the necessary volume of liquid for a single injection (50 μl). The eye was kept open using an eye lid speculum. The needle was inserted in the supero-temporal quadrant, through an area 3.5-4.0 mm posterior to the limbus, avoiding the horizontal meridian and aiming toward the center of the eye. Only half (¼ inch) of the needle total length (½ inch) was inserted into the eye. The injection volume was delivered slowly to prevent a rapid increase in intraocular pressure. To ensure that all the solution is in the eye and to avoid push-back through the injection site, the needle was removed slowly.

Post-injection Procedures: Immediately after the injection, the eye was washed with normal saline solution. Excess liquid on the periocular area was blotted dry with a 4×4 gauze pad. Two drops of antimicrobial ophthalmic solution (Vigamox®, moxifloxacin, 0.5% ophthalmic solution) were then added.

Ophthalmic Examinations: All animals received an ophthalmic exam (indirect ophthalmoscope) by a practicing clinical ophthalmologist prior to sacrifice. The exam scoring key is shown in Table 3.

Euthanasia: Euthanasia was performed on anesthetized rabbits by using a 22 gauge needle for injecting about 2-3 cc of Beuthanasia-D (Schering Plough) through the ear vein.

TABLE 3
Indirect ophthalmoscope exam score keys

| Score | |
|---|---|
| | Vitreous Exam |
| 0 | Clear, no deposits, bubbles, or haziness detectable |
| 1 | Light haziness, no opaque deposits or vitreous opacity |
| 2 | Moderate opacity or opaque bubble or deposit |
| 3 | Pronounced vitreal opacity |
| 4 | Opaque |
| | Retinal Exam |
| 0 | Flat, no mars, blisters or blemishes |
| 1 | Not flat; has wrinkles, bumps, or ripples |
| 2 | Minor inflammation or bleeding |
| 3 | Retinal detachment, moderate hemorrhage |
| 4 | Destruction of retinal tissue (GA, CNV, retinal tear) |

Enucleation of the eyes for preparation of the eye globes: The eye lids were held wide open with one hand and the conjunctiva was cut along the edge of the cornea using a scalpel blade. The external ocular muscles were cut in a clock-wise pattern and the nictating membrane was also cut and removed. The optic nerve was cut with enucleation scissors entering the orbital cavity from the nasal side. The eye globe was picked up “en-block” by dissecting out the structures from the retro-bulbar compartment of the eye. Each enucleated eye globe was trimmed by removing the associate orbital fascia, orbital fat and extra-ocular muscles with the help of a small curved pair of scissors.

Collection of rabbit vitreous fluid, spheroidal intravitreal deposits, and retinal layers from the isolated eye-balls: The trimmed rabbit eye globe was placed in a sterile Petri dish. Grasping the eye with a fine 1×2 tooth forceps, an incision through the sclera gently was made 5 mm behind the limbus with a #11 scalpel blade. Then the crown was separated by cutting the sclera, and retina all the way around the eye. The deposits were carefully picked up with a forceps and then transferred to an eppendorf tubes and kept on ice. If the deposit was too fragile, it was scooped with a #15 scalpel. The vitreous humor was collected by picking it up with the tooth forceps and then transferred to an eppendorf tube and kept on ice. The retinal layer from each eye globe was scrapped from the back of the eye with a #15 scalpel and transferred immediately to an eppendorf tube with RNA Later (Ambion, Austin, Tex.) and kept on ice. All the collected materials (vitreous fluid, diposit and retinal layers) were frozen and stored at −20° C.

Deposit scoring: If a deposit was noticed during material collection, it was scored and collected. The scoring criteria for deposits are shown in Table 4.

TABLE 4
Deposit Scoring Key

| Score | Size | Opacity | Solidity |
|---|---|---|---|
| 0 | No deposit | No deposit | No deposit |
| 1 | ≦1 mm diameter | Clear | Crumbles upon touch or multiple fragile |
| | | | pieces discovered upon globe dissection |
| 2 | 1-2 mm diameter | Blue/white tinge, easily | Easily deformed by forceps; light force |
| | | seen through | permanently deforms deposit(s) |
| 3 | 2-4 mm diameter | Notable blue/white color, | Easily deformed by forceps but elastic; |
| | | not easily seen through | deposit(s) can return to original shape |
| 4 | ≧4 mm diameter | Opaque | Deposit(s) form is fairly firm to forceps |

Histopathological Analysis: After examination, animals were euthanized and eyes were enucleated, fixed in Davidson's solution, sectioned and H/E stained. Sections from both eyes of animals receiving 25, 50, or 100 μg/eye of compound (Table 2, last three animals) were examined by an American Board of Pathology-certified pathologist, and peer-reviewed by a board-certified and recognized expert veterinary pathologist.

HPLC Analysis:

Deposit Sample Preparation

Freeze-dry deposit previous to analysis

Resuspend deposit in 50 μl of 80% acetic acid/20% water

Take 8 μl from gel solution

add 20 μl of the internal standard stock ( 1/16 dilution in Mobile Phase A)

Add 52 μl of Mobile Phase A

Vitreous Sample Preparation

The vitreous sample preparation was performed following SOP-D0002v1 with modifications. This time 20 μl of internal standard (closely related in sequence to the compound) (⅛ dilution in water) were added to the 50 μl of vitreous previous to sample extraction.

Sample Analysis

Both deposit and vitreous samples were analyzed by HPLC using the reverse-phase column; C₁₈ Hypersil Gold AQ, 2.1×150 mm, 5 μm (Thermo Electron), and UV detection at a wavelength of 214 nm. Separate calibration curves were generated for deposit and vitreous analysis.

SDS-PAGE Analysis of Rabbit Intravitreal Deposit Samples:
1. Aliquot rabbit vitreous deposit sample(s) after dissolving freeze-dried deposit in PBS: Acetic Acid (1:1) into eppendorf tube. (deposit samples from animal #1341, 1310A and 1339)
2. Add sample loading dye (4×) and beta mercapto-ethanol.
3. Boil the samples for 5 min over a heating plate.
4. Cool down samples in ice for a few min.
5. Centrifuge the samples at 10,000 rpm for 2 min.
6. Load two volumes (5 ul and 20 ul) of each sample separately in wells of the SDS-PAGE gel leaving one well blank in between.
7. Load SeeBlue protein marker for comparison.
8. Run at 200 constant volts for 1 hour.
9. Remove the gel from the assembly and stain in Coomasie blue dye for 30 min.
10. Cut the visible protein bands from the gel in separate tubes and sent to mass spectrometry facility.

Results

Table 5 presents a summary of results of the study.

TABLE 5
Results of study.

| | | | | | | Gel Scores (after dissection) | | | |
|---|---|---|---|---|---|---|---|---|---|
| Animal ID and Doses | | | | Indirect Exam | | | | | Summed |
| Animal | Eye | Dose | Use | Vit. | Retina | Size | Opacity | Solidity | Score |
| 1342 | Left | 0 | Dissection | 1 | 0 | 0.0 | 0.0 | 0.0 | 0.0 |
| 1342 | Right | 0 | Dissection | 1 | 0 | 0.0 | 0.0 | 0.0 | 0.0 |
| 1312 | Left | 25 | Dissection | 1 | 0 | 0.0 | 0.0 | 0.0 | 0.0 |
| 1312 | Right | 25 | Dissection | 1 | 0 | 0.0 | 0.0 | 0.0 | 0.0 |
| 1334 | Left | 25 | Dissection | 1 | 0 | 0.0 | 0.0 | 0.0 | 0.0 |
| 1334* | Right | 50 | Dissection | 2 | 0 | 1.0 | 1.0 | 0.0 | 2.0 |
| 1335* | Left | 50 | Dissection | 1 | 0 | 0.5 | 0.5 | 0.0 | 1.0 |
| 1335 | Right | 50 | Dissection | 2 | 0 | 0.0 | 0.0 | 0.0 | 0.0 |
| 1344 | Left | 75 | Dissection | 1 | 0 | 0.0 | 0.0 | 0.0 | 0.0 |
| 1344 | Right | 75 | Dissection | 2 | 0 | 0.5 | 0.5 | 0.5 | 1.5 |
| 1345 | Left | 75 | Dissection | 1 | 0 | 0.0 | 0.0 | 0.0 | 0.0 |
| 1345 | Right | 100 | Dissection | 2 | 0 | 0.5 | 0.0 | 0.0 | 0.5 |
| 1346 | Left | 100 | Dissection | 3 | 0 | 0.5 | 1.0 | 0.0 | 1.5 |
| 1346 | Right | 100 | Dissection | 2 | 0 | 0.0 | 0.0 | 0.0 | 0.0 |
| 1310 | Left | 125 | Dissection | 1 | 0 | 0.5 | 0.0 | 0.5 | 1.0 |
| 1313 | Left | 125 | Dissection | 1 | 0 | 2.0 | 1.0 | 1.0 | 4.0 |
| 1313 | Right | 125 | Dissection | 1 | 0 | 2.0 | 2.0 | 2.0 | 6.0 |
| 1310 | Right | 150 | Dissection | 2 | 0 | 2.0 | 2.0 | 2.0 | 6.0 |
| 1338 | Left | 150 | Dissection | 1 | 0 | 3.0 | 3.0 | 3.0 | 9.0 |
| 1338 | Right | 150 | Dissection | 1 | 0 | 2.0 | 3.0 | 3.0 | 8.0 |
| 1339 | Left | 175 | Dissection | 2 | 2 | 1.0 | 1.0 | 1.0 | 3.0 |
| 1339 | Right | 175 | Dissection | 1 | 0 | 2.0 | 0.0 | 2.0 | 4.0 |
| 1340 | Left | 175 | Dissection | 4 | 0 | 1.0 | 1.0 | 1.0 | 3.0 |
| 1340 | Right | 200 | Dissection | 1 | 0 | 2.0 | 2.0 | 2.0 | 6.0 |
| 1341 | Left | 200 | Dissection | 1 | 0 | 3.0 | 3.0 | 2.0 | 8.0 |
| 1341 | Right | 200 | Dissection | 1 | 0 | 3.0 | 3.0 | 3.0 | 9.0 |

*Deposit too small to collect

Indirect examination results and scores are shown in Table 5 along with animal numbers and doses. Deposits were found in all eyes that received a dose equal or higher than 125 μg/eye of compound. No deposits were detected in the eyes that received 25 μg/eye of compound. Deposits were formed at other concentrations at various frequencies. In this study, deposits could not be detected reliably by indirect exam. It should be noted that the ophthalmologist could detect a light haze even at concentrations below those that form deposits.

The indirect ophthalmoscopic exam did not reveal any pathological changes associated with intravitreal compound injection. Fundus exams were normal with the exception of one animal, #1339, which had a retinal hemorrhage that the ophthalmologist attributed to an injury caused by the injection needle. A vitreous haze was noted in 9 of the 32 eyes examined. The presence of a haze did not correlate strongly with compound dose.

Histopathological Evaluation

Five animals in this study were also processed for histopathological evaluation. The animals and the treatments they received are shown in Table 6. The eyes were evaluated by two pathologists. Both pathologists agreed that all the evaluated eyes were normal. The cornea, iris, ciliary body, lens, sclera, retina, and vitreous were all normal.

Analysis of deposits: The deposits were analyzed by HPLC in order to confirm and quantify the presence of compound. An SDS-PAGE analysis was also performed to detect the possible presence of other proteins that could not be identified by the HPLC protocol used (detects mostly low MW peptides). No components other than compound were identified under UV detection. The data confirmed the hypothesis that the compound level present in deposits correlates to the amount of compound injected into the vitreous. The concentration of compound in the rabbit vitreous following intravitreal injection at each dose was measured. After 27 hours of injection there was no observable correlation seen between compound present and compound injected.

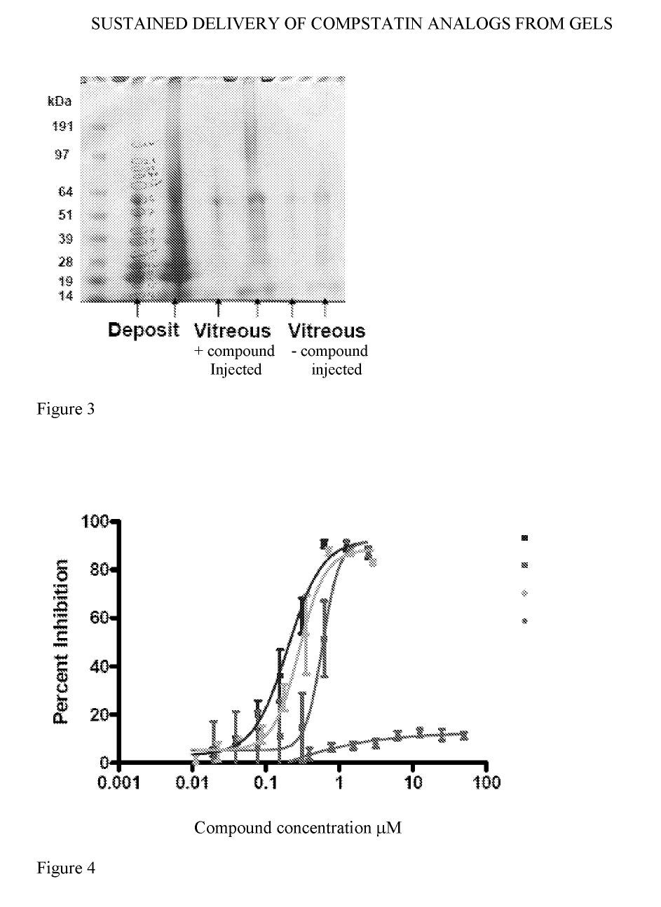

FIG. 3 presents the SDS-PAGE analysis of the deposits from rabbits 1341 right eye (200 ug compound), 1310 right eye (150 ug compound) and 1339 right eye (175 ug compound). The protein bands from lane 2 were cut and sent for identification by mass spectrometery. All visible protein bands from the rabbit vitreous deposit were successfully identified through the MS/MALDI approach. Results are shown in the table below.

| Band | Top Score¹ | Protein ID | Protein Name | Species² |
|---|---|---|---|---|
| 1 | 98 | Q8MIE4_9LAGO | Interphotoreceptor retinoid | Lepus crawshayi |
| | | | binding protein | (Hare) |
| | | | (Fragment). | |
| 2, | 208 | AAB58347 | Serum albumin precursor | Oryctolagus cuniculus |
| mixture | | | | (Rabbit) |
| | 85 | AAB94136 | Serotransferrin precursor | Oryctolagus cuniculus |
| | | | | (Rabbit) |
| 3 | 293 | AAB58347 | Serum albumin precursor | Oryctolagus cuniculus |
| | | | | (Rabbit) |
| 4 | No results | — | — | — |
| | returned | | | |
| 5 | 180 | AAB94136, TFRBP | transferrin precursor | Oryctolagus cuniculus |
| | | | | (Rabbit) |
| 6 | 518 | AAB58347 | Serum albumin precursor | Oryctolagus cuniculus |
| | | | | (Rabbit) |
| 7 | 277 | AAB58347 | Serum albumin precursor | Oryctolagus cuniculus |
| | | | | (Rabbit) |
| 8 | 152 | AAB58347 | Serum albumin precursor | Oryctolagus cuniculus |
| | | | | (Rabbit) |
| | 78 (maybe | CAA27396 | cytoplasmic beta-actin | Mus musculus (mouse) |
| | omit this) | | | |
| 9 | 209 | CRYL1_RABIT | Lambda-crystallin. | Oryctolagus cuniculus |
| | | | | (Rabbit) |
| 10 | 99 | CRBB1_BOVIN | Beta crystallin B1 | Bos taurus (Bovine) |
| 11 | 108 | CRBB1_BOVIN | Beta crystallin B1 | Bos taurus (Bovine) |
| 12 | 203 | CRBB2_HUMAN | Beta crystallin B2 (Beta- | Homo sapiens |
| | | | crystallin Bp) | (Human) |
| 13 | 105 | Q95KK5_RABIT | Beta A1-crystallin | Oryctolagus cuniculus |
| | | | | (Rabbit) |
| 14 | 167 | CYRBAA | alpha-crystallin chain A | Oryctolagus cuniculus |
| | | | | (Rabbit) |
| 15 | 97 | CYRBAA | alpha-crystallin chain A | Oryctolagus cuniculus |
| | | | | (Rabbit) |
| 16³ | 105 | CYRBAA | alpha-crystallin chain A | Oryctolagus cuniculus |
| mixture | | | | (Rabbit) |

¹Mascot Search from www.matrixscience.com. This is the most likely identity of the protein analyzed by MALDI-MS. Higher scores reflect higher confidence levels.
²If highest score not rabbit, it is likely the rabbit version is not in database
³The other two components for the mixture were identified as CAA42911 (RRHARTABC) from Rattus rattus (score 81) and HBB_RABIT (Hemoglobin subunit beta-1/2 ) from Oryctolagus cuniculus (Rabbit).

In conclusion, deposit formation was dose-dependant. Deposits were never observed to form at doses of 25 μg/eye, and reliably formed in eyes receiving a dose of 125 μg/eye or higher (all in a volume of 50 μl). Analysis of the spheroidal gel-like intravitreal deposits showed active compound to be a major component of the deposit, and also that the protein content of the deposits is limited to proteins present in the vitreous humor of rabbits. Histopathological evaluations were performed on eyes of rabbits receiving single-dose intravitreal injections of 25, 50, and 100 μg/eye compound 24 hours post injection. Both pathologists agreed all examined eye sections were normal.

In a subsequent study New Zealand white rabbits were maintained for more than 8 months following intravitreal administration of compound. Compound extracted from gels removed from the rabbit vitreous at times ranging from 6 weeks to 8 months following administration remained stable and retained substantial complement inhibiting activity in standard assays (FIG. 4).

In other work, rabbits were retreated 3 months after initial treatment (following apparent disappearance of the gel-like deposit formed following initial treatment). The second treatment resulted in formation of a new deposit, confirming the feasibility of repeated treatment using this modality for sustained release.

### Example 3

### Further Studies of Compstatin Analog Deposits in Non-Human Primates

To further explore the behavior of thedeposits in the eye of non-human primates over time, additional studies were performed. As in the rabbits, it was found that deposits could be visualized under ophthalmoscopic examination and tracked over time using ultrasound.

In one study, Cynomolgus monkeys were administered 0, 150, 450, 1050, or 2100 μg of compound in 50 μl WFI by intravitreal injection. It was noted that 2 weeks following injection deposits were visible in all the eyes that had been given the 150 μg dose and also in the eyes that had been administered a higher dose.

Some of the animals that had been administered 0, 450, 1050, or 2100 μg were sacrificed, and compound concentration in serum and vitreous was measured 14 days following administration. Compound measurement was performed using HPLC. As shown in FIG. 5, the compound is slowly released from intravitreal deposits in monkeys for at least two weeks. When administered at concentrations too low to form a deposit, the compound is cleared from the Cynomolgus vitreous with a T_{1/2} of about 22 hours. Therefore, it is evident that the compound measured in this experiment after 2 weeks must reflect release from the deposit rather than residual compound that was never present in the deposit.

The study was continued to evaluate behavior of the deposits over time. Deposits formed at the 150 μg dose remained detectable after 2 months but decreased in size. Some were completely gone at 3 months while some persisted for as long as 6 months. Deposits formed at doses of 450, 1050, or 2100 μg remained detectable after 6 months. There was evidence that they were diminishing in size and/or density at the 6 month time point. Deposits formed at the 450, 1050, and 2100 μg dose levels continued, in most cases, to be observable at the 37 week time point and up to the 1 year time point at which the study ended. The deposits could be observed both by ophthalmoscopic examination and ultrasound. They appeared to continue diminishing in size and/or split into multiple smaller deposits over time. Furthermore, continued release of compstatin analog occurred during this time, as determined based on measuring compstatin analog in serum samples obtained from these animals.

In an independent study, gel-like deposits were observed both by ophthalmoscopic examination and ultrasound when compound in water was administered by intravitreal injections at concentrations as low as 1 mg/ml (50 μg in 50 μl). The deposits were undetectable using these techniques by approximately 1 week following dosing.

It was noted that overall, formation and rate of disappearance of the deposits was more reproducible in the monkeys than had been found in rabbits. In general, it was noted that the deposits formed in rabbits are denser and last longer than the same doses administered to monkeys. No adverse effects attributable to the deposits were observed either histopathologically or during physical examination in either the monkey or rabbit studies.

### Example 4

### Modulation of Deposit Properties

Various excipients, buffers, and pH ranges were tested to assess their potential to modify the gel-forming properties of the compstatin analog used in Examples 1-3 and to assess their effects on the properties of the gels in vitro an in vivo. Amino acids, including arginine, serine, and histidine were assessed. Formulations containing histidine were found to exhibit favorable properties in terms of gel stability relative to those containing arginine or serine.

In some experiments, the effect of various concentrations of sodium acetate (NaCH₃COO), histidine, and mannitol either individually or in various combinations were tested in vitro and/or in vivo. Concentrations of histidine and mannitol ranged from 10 to 50 mM. In general, it was observed that formulations that contained any of these materials resulted in gels that were more fragile and decreased in size more rapidly in vivo than gels formed from formulations containing only compstatin analog in water. It was concluded that addition of these excipients permits modulation of the rate of size diminution and, as a result, modulation of the rate of release of compstatin analog from the deposit. Such modulation would potentially allow the administration of a higher total dose of compstatin analog

Exemplary formulations included the following:

A. 100 mM solution of sodium acetate (pH=5.10) in water for injection.

B. 100 mM Sodium Acetate+25 mM Histidine (pH=5.2)

C. 100 mM Sodium Acetate+45 mM Mannitol (pH=5.05)

Range of pH˜(5-5.50)

Materials:

### 1. Sodium Acetate:

Stock solution: 3 M
Manufacturer: Ambion
pH=5.5
Final concentration in the formulation: 100 mM
Final pH of formulation: 5.1

### 2. Histidine:

DL-Histidine hydrochloride, 98% min
Molecular weight: 209.64
Manufacturer: Acros Organics

### 3. D-Mannitol

USP powder
Manufacturer: Fisher Scientific

Protocol for Formulation:
For making 10 ml of solution:
1. Dilute sodium acetate in Di water from 3 M to 100 mM.
2. Weigh exact amount of histidine/mannitol to reach desired concentration.
3. Determine final pH and osmolarity of the solution.
For making desired formulation of compstatin analog:
1. Weigh empty tubes
2. Add desired amount of compstatin analog into those tubes.
3. Add solution (water/sodium acetate/sodium acetate+mannitol/sodium acetate+histidine) to reach desired concentration of compstatin analog
4. Filter the solution by 0.22 micron sterile filter
5. Measure the final absorbance to verify final concentration of compstatin analog
6. Measure pH of the final solution.

In some experiments, compstatin analog was added to a 100 mM solution of sodium acetate (pH=5.10) in water for injection. The formulation (1050 μg compstatin analog in 50 μl liquid) was administered by intravitreal injection to 3 non-human primates. All 3animals exhibited deposits at weeks 2 through 6. As of week 9, no significant deposits were observed. One of the 3 animals exhibiting a remnant. In contrast, animals who had received equal amounts of compstatin analog formulation consisting of compstatin analog dissolved in WFI exhibited deposits at week 9. Presence of sodium acetate thus apparently increased the rate of dissolution of the deposits.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is not limited by particular results achieved in any specific example or with any specific embodiment. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more elements or limitations found in any other claim that is dependent on the same base claim. Furthermore, where the claims recite a composition, it is to be understood that methods of administering the composition according to any of the methods disclosed herein, and methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. For purposes of conciseness only some of these embodiments have been specifically recited individually and specifically herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

The inclusion of a “providing” step in certain methods of the invention is intended to indicate that the composition is administered to treat a disorder recited in the method. Thus the subject will have or be at risk of the disorder and the composition is administered to treat the disorder, typically upon recommendation of a medical or surgical practitioner, who may or may not be the same individual who administers the composition. The invention includes embodiments in which a step of providing is not explicitly included and embodiments in which a step of providing is included. The invention also includes embodiments in which a step of identifying the subject as being at risk of or suffering from a complement-mediated disorder is included.

Where ranges are given, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. A time period of 1 month is understood to mean 30 days. A time period of 1 year is understood to mean 365 days. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

It is to be understood that any particular embodiment, feature, or aspect of the present invention may be explicitly excluded from any one or more of the claims. For example, any particular composition, compound or class of compounds, site of administration, route or method of administration, dose, formulation, or complement-mediated disorder can be excluded from any one or more claims.

1. A method of treating a complement-mediated disorder comprising the step of: administering a liquid composition comprising an effective amount of a compstatin analog to an extravascular location of a subject, wherein said effective amount is sufficient to form a discrete, macroscopic gel-like structure containing the compstatin analog within said extravascular location.

2. The method of claim 1, wherein said effective amount is sufficient to form a macroscopic gel-like structure that diminishes in size over time and remains readily detectable for a period of time selected from the group consisting of: at least 2 weeks or at least 3 months.

3. The method of claim 1, wherein said effective amount is sufficient to form a macroscopic gel-like structure that diminishes in size over time and releases compstatin analog in active form for a period of time selected from the group consisting of: at least 2 weeks or at least 3 months.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A gel-like structure comprising a compstatin analog and at least one endogenous polypeptide normally present in an extravascular location of a subject.

26. The gel-like structure of claim 24, wherein the extravascular location is selected from the group consisting of: the vitreous chamber, subconjunctival space, sub-Tenon's space, subretinal space, synovial cavity, and the cerebrospinal cavity.

27. A liquid composition comprising a compstatin analog, wherein the composition is characterized in that it forms a macroscopic, gel-like structure when administered to the vitreous chamber of a mammalian subject.

28. The liquid composition of claim 27, wherein the compstatin analog in present in an amount sufficient to form a discrete, macroscopic gel-like structure when administered to the vitreous chamber of the subject by intravitreal injection.

29. The liquid composition of claim 27, wherein the compstatin analog is present in an amount sufficient to form a macroscopic gel-like structure that diminishes in size over time and remains readily detectable for a period of time selected from the group consisting of: at least 2 weeks, at least 3 months, or at least 6 months.

30. The liquid composition of claim 27, wherein the compstatin analog is present in an amount sufficient to form a macroscopic gel-like structure that diminishes in size over time and releases compstatin analog in active form for a period of time selected from the group consisting of: at least 2 weeks, at least 3 months, or at least 6 months.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. The liquid composition of claim 27, wherein the composition comprises a component selected from the group consisting of: polyols and amino acids.

55. The liquid composition of claim 53 wherein the presence of the component modulates the rate at which the deposit disappears in vivo.

56. (canceled)

57. (canceled)

58. (canceled)

59. (canceled)

60. The liquid composition of claim 27, wherein the liquid composition further comprises an effective amount of a second therapeutic agent.

61. The liquid composition of claim 59, wherein the second therapeutic agent is a complement inhibitor, angiogenesis inhibitor, steroid, anti-inflammatory agent, anti-infective, or analgesic.

62. The liquid composition of claim 59, wherein the second therapeutic agent is an antibody or antibody fragment.

63. A method of preparing a composition for delivery of a therapeutic agent over a sustained period of time comprising: preparing a liquid composition comprising the therapeutic agent and a compstatin analog, wherein the compstatin analog is present in sufficient amounts to form a macroscopic, gel-like structure when the composition is administered to an extravascular location of a mammalian subject.

64. (canceled)

65. (canceled)

66. (canceled)

67. A method of treating a subject suffering from or at risk of AMD comprising administering a liquid composition comprising a compstatin analog directly to the subject's vitreous chamber, wherein the liquid composition contains a sufficient amount of the compstatin analog to form a macroscopic, gel-like structure following administration.

68. The method of claim 1 or 67, wherein the compstatin analog comprises a peptide whose sequence comprises a sequence selected from the group consisting of SEQ ID NOs: 3, 4, 5, 6, and 7.

69. The method of claim 1 or 67, wherein the compstatin analog has at least 100-fold greater activity than SEQ ID NO: 8.

70. The method of claim 1 or 67, wherein the compstatin analog has at least 200-fold greater activity than SEQ ID NO: 8.

71. The method of claim 1 or 67, wherein the compstatin analog has a sequence selected from SEQ ID NOs: 14, 21, 28, 29, 30, 31, 32, 33, 34, and 36.

72. (canceled)

73. (canceled)

74. (canceled)

75. (canceled)

76. (canceled)

77. (canceled)

78. A liquid composition comprising a compstatin analog and water, wherein the concentration of the compstatin analog is between 3 and 50 mg/ml.

79. The liquid composition of claim 77, wherein the concentration of the compstatin analog is between 5 and 30 mg/ml.

80. The liquid composition of claim 77, wherein the concentration of the compstatin analog is between 8 and 25 mg/ml.

81. The liquid composition of any of claims 27 or 77, wherein the compstatin analog comprises a peptide selected from the group consisting of SEQ ID NOs: 3, 4, 5, 6, and 7.

82. The liquid composition of any of claims 27 or 77, wherein the compstatin analog has at least 100-fold greater activity than SEQ ID NO: 8.

83. The liquid composition of any of claims 27 or 77, wherein the compstatin analog has at least 200-fold greater activity than SEQ ID NO: 8.

84. The liquid composition of any of claims 27 or 77, wherein the compstatin analog has a sequence selected from SEQ ID NOs: 14, 21, 28, 29, 30, 31, 32, 33, 34, and 36.

85. (canceled)

86. (canceled)

87. (canceled)
